(19) United States
(12) Patent Application Publication
(10) Pub. No.: US 2024/0197155 A1
(43) Pub. Date: Jun. 20, 2024

(54) SYSTEMS, DEVICES, AND METHODS FOR PROVIDING MOTORIZED CONTROL FOR MEDICAL DEVICES

(71) Applicant: Boston Scientific Scimed, Inc., Maple Grove, MN (US)

(72) Inventors: Martin FAWDRY, Galway (IE); Aiden FLANAGAN, Co. Galway (IE); Michael HUGHES, Galway (IE); Anthony O'BRIEN, Galway (IE); Pearse COFFEY, Galway (IE); Stephen McCOOEY, Dundalk (IE); Leo DOWNES, Co. Galway (IE); Matthew LYNSKEY, Galway (IE); Donal HUNT, Galway (IE)

(73) Assignee: Boston Scientific Scimed, Inc., Maple Grove, MN (US)

(21) Appl. No.: 18/541,422

(22) Filed: Dec. 15, 2023

Related U.S. Application Data
(60) Provisional application No. 63/512,240, filed on Jul. 6, 2023.
(60) Provisional application No. 63/387,775, filed on Dec. 16, 2022.

(51) Int. Cl. A61B 1/00; A61B 1/005

(52) U.S. Cl. CPC A61B 1/0016; A61B 1/00045; A61B 1/00131; A61B 1/0053

(57) ABSTRACT

A motorized control system for a medical device may include a control assembly including a first body with a cradle assembly and a second body moveably coupled to the first body. The cradle assembly may be configured to removably couple to the medical device. The second body may include a gear assembly, a first motor configured to drive a first gear of the gear assembly, and a second motor configured to drive a second gear of the gear assembly. The control assembly may be configured to transition between an open configuration and a closed configuration. The gear assembly may be configured to receive a plurality of knobs of the medical device in the closed configuration. The first motor may be configured to drive the first gear to rotate a first knob. The second motor may be configured to drive the second gear to rotate a second knob.

### CROSS-REFERENCE TO RELATED APPLICATIONS

This application claims the benefit of priority of U.S. Provisional Patent Application No. 63/387,775, filed on Dec. 16, 2022, and U.S. Provisional Patent Application No. 63/512,240, filed on Jul. 6, 2023, each of which is incorporated herein by reference in its entirety.

### TECHNICAL FIELD

This disclosure relates generally to motorized controls for medical devices and related accessories. More particularly, in embodiments, the disclosure relates to a system, devices, and methods that allows switching between manual and motorized control of an endoscope or other medical device, among other aspects.

### BACKGROUND

Medical devices for accessing target sites within a body may be advanced through one or more lumens of the body, and such medical devices are typically manually controlled by a user. For example, in many endoscopes, the distal end of the endoscope may be steered by knobs on the proximal end, and a user may struggle to control the knobs while navigating altered anatomy. This often creates difficulties in maneuvering the endoscope to target sites, or performing other therapies, as a user may not be able to maintain precise control of the endoscope with their hands during a long procedure. However, a fully motorized steering system for an endoscope may create difficulties for a user when performing portions of a procedure, such as the initial insertion of the endoscope into the patient or navigation to a target site. A user may prefer to steer the endoscope manually or to steer the endoscope robotically using a motorized system during more difficult portions of a procedure.

This disclosure may solve one or more of these problems or other problems in the art. The scope of the disclosure, however, is defined by the attached claims and not the ability to solve a specific problem.

### SUMMARY

Aspects of the disclosure relate to, among other things, systems, devices, and methods for providing motorized control for an endoscope or other medical device.

In some aspects, a motorized control system for a medical device may include a control assembly that may include a first body including a cradle assembly and a second body moveably coupled to the first body. The cradle assembly may be configured to removably couple to the medical device. The second body may include a gear assembly, a first motor configured to drive a first gear of the gear assembly, and a second motor configured to drive a second gear of the gear assembly. The control assembly may be configured to transition between an open configuration and a closed configuration. The gear assembly may be configured to receive a plurality of knobs of the medical device in the closed configuration. The first motor may be configured to drive the first gear to rotate a first knob of the plurality of knobs. The second motor may be configured to drive the second gear to rotate a second knob of the plurality of knobs.

The motorized control system may include one or more of the following aspects. The motorized control system may further include a rotary drive coupled to a proximal end of the control assembly, and the rotary drive may be configured to rotate the control assembly and the medical device about a longitudinal axis of the medical device. The motorized control system may further include a base assembly coupled to the rotary drive, and a rail assembly coupled to the base assembly. The rail assembly may include at least one motor, and may be configured to move the control assembly in proximal and distal directions. The control assembly may further include an actuator, and the actuator may include an elevator actuator configured to align with an elevator lever of the medical device, an elevator motor, a third gear coupled to the elevator motor, and a rack engaged with the third gear. The actuator assembly may be configured to move an elevator lever of the medical device.

The second body may further include a first worm gear coupled to the first motor and engaged with the first gear, and a second worm gear coupled to the second motor and engaged with the second gear. The first motor, the first worm gear, and the first gear may be longitudinally aligned. The second motor, the second worm gear, and the second gear may be longitudinally aligned. The first gear may be adjacent to the second gear. The first gear may include a series of recesses configured to align with prongs of the first knob, and the second gear may include a series of recesses configured to align with prongs of the second knob. The control assembly may be controlled by a control unit including an electronic display. The first body may include a first rail portion. The first rail portion may be extendable outward from the first body and retractable inward into the first body, and the second body may be fixedly coupled to the first rail portion.

The cradle assembly may include a U-shaped portion and a gate rotatably coupled to the U-shaped portion. The motorized control system may further include a telescopic support assembly coupled to a distal end portion of the cradle assembly. The first gear may include a first plurality of spring-biased pins configured to engage the first knob, and the second gear may include a second plurality of spring-biased pins configured to engage the second knob. The motorized control system may further include a remote control configured to communicate with the control assembly to operate the first motor and the second motor. The medical device may be an endoscope. The second body may further include a camera system configured to detect a position of the first knob, a position of the second knob, and a position of an elevator. In some other aspects, a motorized control system for a medical device may include a control assembly, a rotary drive, and a rail assembly. The control assembly may include a first body including a cradle assembly, and a second body moveable coupled to the first body. The cradle assembly may be configured to removably couple to the medical device. The second body may include a gear assembly and a first motor configured to drive a first gear of the gear assembly. The rotary drive may be coupled to a proximal end of the control assembly, and the rotary drive may be configured to rotate the control assembly and the medical device about a longitudinal axis of the medical device. The rail assembly may be coupled to the rotary drive, and the rail assembly may include at least one motor configured to drive movement of the rotary drive in a proximal or distal direction. The control assembly may be configured to transition between an open configuration and a closed configuration. The gear assembly may be configured to receive a first knob of the medical device in the closed configuration. The first motor may be configured to drive the first gear to rotate the first knob.

The motorized control system may include one or more of the following aspects. The control assembly may further include an actuator. The actuator may include an elevator actuator configured to align with an elevator lever of the medical device, an elevator motor, a third gear coupled to the elevator motor, and a rack engaged with the third gear. The actuator assembly may be configured to move an elevator lever of the medical device. The first gear may include a series of recesses configured to align with prongs of the first knob.

In additional aspects, a motorized control system for a medical device may include a control assembly with a first body, a second body, and an actuator assembly. The first body may be configured to removably couple to the medical device. The second body may be moveably coupled to the first body. The second body may include a gear assembly and a first motor configured to drive a first gear of the gear assembly. The actuator assembly may be configured to move an elevator lever of the medical device. The actuator assembly may include an elevator actuator configured to align with an elevator lever of the medical device, an elevator motor, a third gear coupled to the elevator motor, and a rack engaged with the third gear. The control assembly may be configured to transition between an open configuration and a closed configuration. The gear assembly may be configured to receive a first knob of the medical device in the closed configuration. The first motor may be configured to drive the first gear to rotate the first knob.

The second body of the motorized control system may further include a first worm gear coupled to the first motor and engaged with the first gear.

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary aspects of the disclosure and together with the description, serve to explain the principles of the disclosure.

FIG. 1 is a perspective view of a motorized control system coupled to an endoscope, according to aspects of this disclosure.

FIG. 2 is a perspective view of a portion of the motorized control system of FIG. 1 coupled to an endoscope, according to aspects of this disclosure.

FIG. 3 is a perspective view of a portion of the motorized control system of FIG. 1, according to aspects of this disclosure.

FIG. 4 is a perspective view of a portion of the motorized control system of FIG. 1, according to aspects of this disclosure.

FIGS. 5A and 5B are side and top views of a portion of the motorized control system of FIG. 1, according to aspects of this disclosure.

FIG. 6 is a side view of a portion of the motorized control system of FIG. 1, according to aspects of this disclosure.

FIG. 7 is a perspective view of a portion of the motorized control system of FIG. 1 and an endoscope, according to aspects of this disclosure.

FIGS. 8-10 are different perspective views of a portion of the motorized control system of FIG. 1 and an endoscope, according to aspects of this disclosure.

FIGS. 11 and 12 are a perspective view and a magnified view of another motorized control system, according to aspects of this disclosure.

FIGS. 13A, 13B, 14, and 15 are various views of an accessory device, according to aspects of this disclosure.

FIG. 16 is a side view of a portion of a motorized control system, according to aspects of this disclosure.

FIG. 17 is a simplified functional block diagram of a computer and/or server that may be configured as a device or system performing any of the methods described herein, according to aspects of this disclosure.

FIG. 18 is a perspective view of a motorized control system coupled to an endoscope, according to aspects of this disclosure.

FIGS. 19 and 20 are side views of a portion of the motorized control system of FIG. 18 coupled to an endoscope, according to aspects of this disclosure.

FIG. 21 is a perspective view of a portion of the motorized control system of FIG. 18 coupled to an endoscope, according to aspects of this disclosure.

FIG. 22 is a perspective view of a portion of the motorized control system of FIG. 18, according to aspects of this disclosure.

FIG. 23 is a perspective view of a portion of an actuator of the motorized control system of FIG. 18, according to aspects of this disclosure.

FIG. 24 is a perspective view of a motorized control system, according to aspects of this disclosure.

FIG. 25 is a perspective view of a motorized control system coupled to an endoscope, according to aspects of this disclosure.

FIG. 26 is a perspective view of a motorized control system coupled to an endoscope, according to aspects of this disclosure.

FIG. 27 is a side view of a portion of the motorized control system of FIG. 26, according to aspects of this disclosure.

FIGS. 28-34 illustrate various views of an exemplary actuator assembly for a medical device, according to aspects of this disclosure.

### DETAILED DESCRIPTION

This disclosure describes exemplary medical systems, methods, and medical tools for controlling a medical device, for example, for controlling movement and operation of an endoscope. This may provide improved medical device functionality and/or assist medical professionals with maneuvering a medical device for performing medical procedures. However, it should be noted that reference to any particular device and/or any particular procedure is provided only for convenience and not intended to limit the disclosure. A person of ordinary skill in the art would recognize that the concepts underlying the disclosed systems, devices, and application methods may be utilized in any suitable procedure, medical or otherwise.

Reference will now be made in detail to aspects of this disclosure, examples of which are illustrated in the accompanying drawings. Wherever possible, the same or similar reference numbers will be used through the drawings to refer to the same or like parts. The term “distal” refers to a portion farthest away from a user when introducing a device into a patient. By contrast, the term “proximal” refers to a portion closest to the user when placing the device into the patient. Throughout the figures, arrows labeled “P” and “D” are used to show the proximal and distal directions in the figures. As used herein, the terms “comprises,” “comprising,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Further, relative terms such as, for example, “about,” “substantially,” “approximately,” etc., are used to indicate a possible variation of ±10% in a stated numeric value or range.

Systems, related devices, and methods, of this disclosure may provide a motorized and/or robotic system which is capable of allowing a user (e.g., an endoscopist, etc.) to reproduce the manual range of motions in an endoscope and the endoscope's articulable distal section to carry out diagnostic and therapeutic procedures, such as endoscopic retrograde cholangiopancreatography (ERCP) procedures. Embodiments of this disclosure seek to improve the control of a medical device, such as an endoscope, during a medical procedure. As non-limiting exemplary benefits, aspects of this disclosure may reduce the total procedure time, may reduce operator fatigue during a medical procedure, may improve the stability, control and/or precision of endoscope movements, among other aspects. The systems, devices, and methods of this disclosure may provide a user with a choice of fully motorized control of a medical device or manual control of the medical device, and may enable a user to switch between fully motorized control of the medical device and manual control of the medical device intraoperatively.

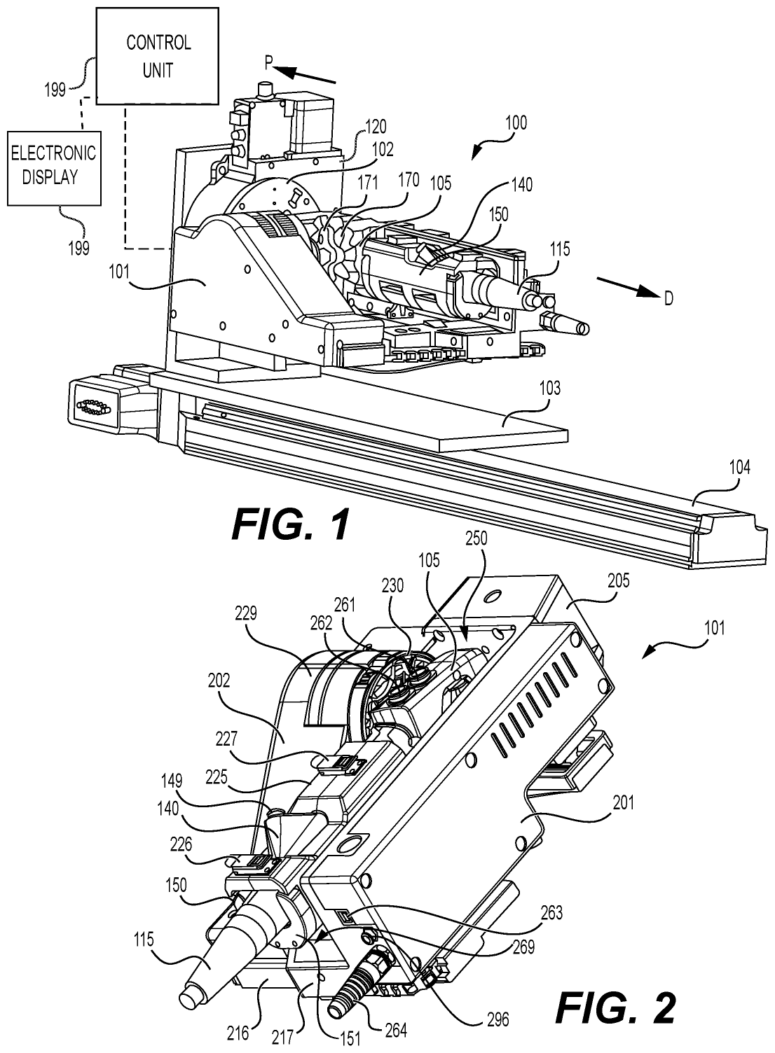

Referring to FIG. 1, a motorized control system 100 in accordance with one or more embodiments of this disclosure is shown. Motorized control system 100 may be configured to couple to a medical device, such as endoscope 105, via cradle assembly 150. A control assembly 101 may be coupled to cradle assembly 150, and may at least partially surround cradle assembly 150. Control assembly 101 may be coupled at its proximal end to a rotary drive 102 and a base assembly 120, and may be connected to a control unit 199 (via one or more wires, wirelessly, or any other means of electrical connection). Rotary drive 102 and base assembly 120 may be coupled to a base plate 103, and base plate 103 may be moveably coupled to a rail assembly 104. Although not shown, motorized control system 100 may include a motor and a gear assembly, for example, instead of or in addition to rotary drive 102. As will be discussed in detail herein below, control system 100 may be configured to control movement of endoscope 105 in a proximal and distal direction, control rotation of endoscope 105 (including a shaft of endoscope, not shown) about a central longitudinal axis in both clockwise and counter-clockwise directions, control movement of a distal articulation section of a shaft of endoscope 105 via rotation of knobs 170, 171, control movement of an elevator of endoscope 105 via movement of one or more levers of endoscope 105, and/or control actuation of one or more suction lumens or water jets of endoscope 105.

Endoscope 105 is shown with its longitudinal shaft and distal tip removed for illustration purposes only, however any endoscope shaft and distal tip structure known in the art may be incorporated in endoscope 105. For example, a shaft may be coupled to a distal end 115 of handle assembly 140. Although the term endoscope may be used herein, it will be appreciated that other devices, including, but not limited to, duodenoscopes, colonoscopes, ureteroscopes, bronchoscopes, laparoscopes, sheaths, catheters, or any other suitable delivery device or other type of medical device may be used in connection with the systems, devices, and methods of this disclosure, and the systems, devices, and methods discussed below may be incorporated into any of these or other medical devices.

Endoscope 105 may include a handle assembly and a flexible tubular shaft (not shown). The handle assembly may include one or more of a biopsy port, an image capture button, an elevator lever/actuator 1704 (FIG. 7), a locking lever, a locking knob, a first control knob 170, a second control knob 171, a suction button, an air/water button, a handle assembly/body 140, and an umbilicus. All of the actuators, elevators, knobs, buttons, levers, ports, or caps of endoscope 105, such as those enumerated above, may serve any purpose and are not limited by any particular use that may be implied by the respective naming of each component used herein. The umbilicus (not shown) may extend from handle assembly 140 to one or more auxiliary devices, such as control unit 199, water/fluid supply, and/or vacuum source. The umbilicus may transmit signals between endoscope 105 and control unit 199, in order to control lighting and imaging components of endoscope 105 and/or receive image data from endoscope 105. The umbilicus also can provide fluid for irrigation from a water/fluid supply and/or suction to a distal tip of a shaft of endoscope 105. Buttons 261 and 262 (FIG. 2) may control valves for suction and fluid supply (e.g., air and water), respectively. A shaft of endoscope 105 may terminate at a distal tip, and the shaft may include an articulation section for deflecting the distal tip in up, down, left, and/or right directions. Knobs 170 and 171 may be used for controlling such deflection.

The distal tip of endoscope 105 may include one or more imaging devices and lighting sources (e.g., one or more LEDs, optical fibers, and/or other illuminators). Examples of imaging devices (or viewing elements) include one or more cameras, one or more image sensors, endoscopic viewing elements, optical assemblies including one or more image sensors and one or more lenses, and any other imaging device known in the art. Distal tip of endoscope 105 may include an elevator for moving an accessory device at the distal tip of endoscope 105, and handle assembly 140 may include a lever for actuation of the elevator (such as elevator lever/actuator 1704).

Control unit 199 may be capable of interfacing with endoscope 105 to provide power and instructions for imaging devices and illuminators. Control unit 199 may also control other aspects of endoscope 105, such as, for example, the application of suction, the deployment or delivery of fluid, and/or the movement of a distal tip of endoscope 105. Control unit 199 may be powered by an external source such as an electrical outlet, and/or may be battery powered. In addition, control unit 199 may include one or more buttons, knobs, touchscreens, or other user interfaces to control the imaging devices, illuminators, and other features of endoscope 105. In some examples, endoscope 105 and control system 100 may be electrically connected, via a wireless communication and/or one or more wires, to the same control unit 199. In other examples, endoscope 105 may be connected to a different control unit 199 from control system 100. In some examples, control system 100 may include a control unit 199 incorporated into a portion of control system 100. In some examples, endoscope 105 may be connected to control unit 199 via an umbilicus.

Control unit 199 may include electronic circuitry configured to receive, process, and/or transmit data and signals between endoscope and one or more other devices, such as control system 100. For example, control unit 199 may be in electronic communication with a display configured to display images based on image data and/or signals processed by control unit 199, which may have been generated by the imaging devices of endoscope 105. Control unit 199 may be in electronic communication with the display in any suitable manner, either via wires or wirelessly. The display may be manufactured in any suitable manner and may include touch screen inputs and/or be connected to various input and output devices such as, for example, mouse, electronic stylus, printers, servers, and/or other electronic portable devices. Control unit 199 may include software and/or hardware that facilitates operations such as those discussed above. For example, control unit 199 may include one or more algorithms, models, or the like for executing any of the methods and/or systems discussed in this disclosure.

When operating endoscope 105 manually, a user may control the movement of a distal end of a shaft of endoscope 105 using their left hand to manipulate knobs 170, 171, in addition to locking levers for each knob 170, 171. The user may also rotate endoscope 105 about its longitudinal axis using their left hand. The insertion and withdrawal of the shaft of endoscope 105 into a patient may be controlled by the user's right hand, left hand, or both hands. Control system 100 may be configured to provide motorized control of the movement and operation of endoscope 105.

Referring again to FIG. 1, endoscope 105 may be fixedly coupled to control assembly 101 during operation of control system 100. Control assembly 101, rotary drive 102, and base assembly 120 may move proximally or distally via base plate 103 moving relative to rail assembly 104, which may move endoscope 105 proximally or distally, respectively. Base plate 103 may be moved relative to rail assembly 104 via a motor, for example, a motor incorporated into rail assembly 104 and/or base plate 103. Endoscope 105 may be rotated about a longitudinal axis via rotation of rotary drive 102, which may rotate control assembly 101 and endoscope 105. Control assembly 101 may include motors 401, 402 (FIG. 4) configured to rotate knobs 170, 171 of endoscope 105 to deflect a distal tip of endoscope 105. Details of the features and operation of control assembly 101 will be discussed herein below with regard to FIGS. 2-10.

FIG. 2 illustrates a perspective view of control assembly 101 coupled to endoscope 105. Control assembly 101 may include a first body 201 and a second body 202 slideably received by the first body 201. Cradle assembly 150 may be coupled to first body 201, and cradle assembly 150 may be configured to receive a medical device, such as endoscope 105. Cradle assembly 150 may include a gate 225 and a cradle body 151. Cradle body 151 may be U-shaped and may include a radially-inward facing surface configured to align with an exterior body of handle assembly 140 of endoscope 105. The shape of the radially-inward facing surface of cradle body 151 may only allow handle assembly 140 to be received within cradle body 151 in the correctly oriented position for operation of control system 100, and thus simplifies the process of positioning handle assembly 140 within cradle body 151. In some examples, cradle assembly 150 may include one or more markers/pointers/indications that are configured to align with endoscope landmarks, such as a centerline of handle assembly 140 or a joint line between a strain relief portion of handle assembly 140 and a body of handle assembly 140. For example, endoscope 105 and/or first body 201 may include one or more physical protrusions and/or one or more visible markers/pointers/indications, which may help in the positioning of handle assembly 140 within cradle body 151. In some aspects, gate 225 may include one or more locating pins, and the one or more locating pins may be inserted into or otherwise align with one or more screw recesses on the handle of endoscope 105, for example, to help ensure proper positioning of handle assembly 140 within cradle body 151.

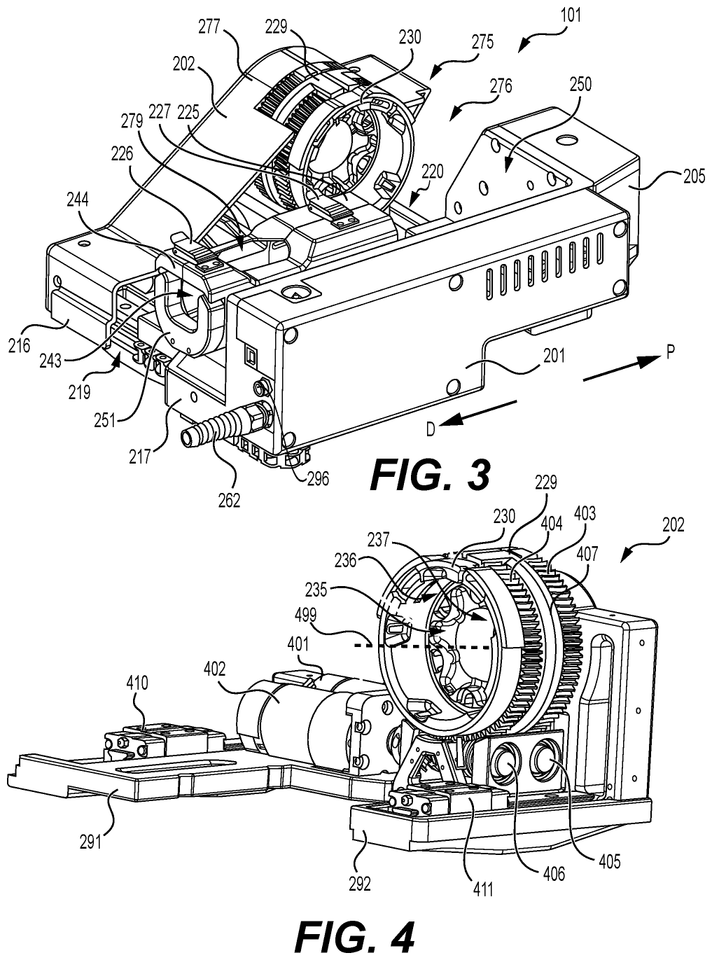

FIG. 3 illustrates a perspective view of control assembly 101 without endoscope 105. A distal end 251 of cradle body 151 may be aligned with a distal portion 217 of first body 201. Gate 225 may be rotatably coupled to cradle body 151, and a distal end 244 of gate 225 may be aligned with distal end 251 of cradle body 151. Gate 225 may rotate about a hinge extending longitudinally across cradle body 151, and rotation of gate 225 may open gate 225 to provide access to a channel 243 extending longitudinally through cradle body 151. In some examples, gate 225 may include one or more fasteners 226, 227 for locking gate 225 to cradle body 151. For example, a user may rotate gate 225 to an open position to provide access to channel 243, insert endoscope 105 into channel 243, and then rotate gate 225 to a closed position and lock gate 225 in a closed position (e.g., using fasteners 226, 227). After locking gate 225 in a closed position with endoscope 105 positioned within channel 243, endoscope 105 may be fixedly coupled to first body 201. Gate 225 may include an opening 279 configured to receive a working channel port/biopsy port 149 of endoscope 105. Cradle assembly 150 may be configured to be coupled to handle assembly 140 at a position distal from knobs 170, 171 and proximal from distal end 115.

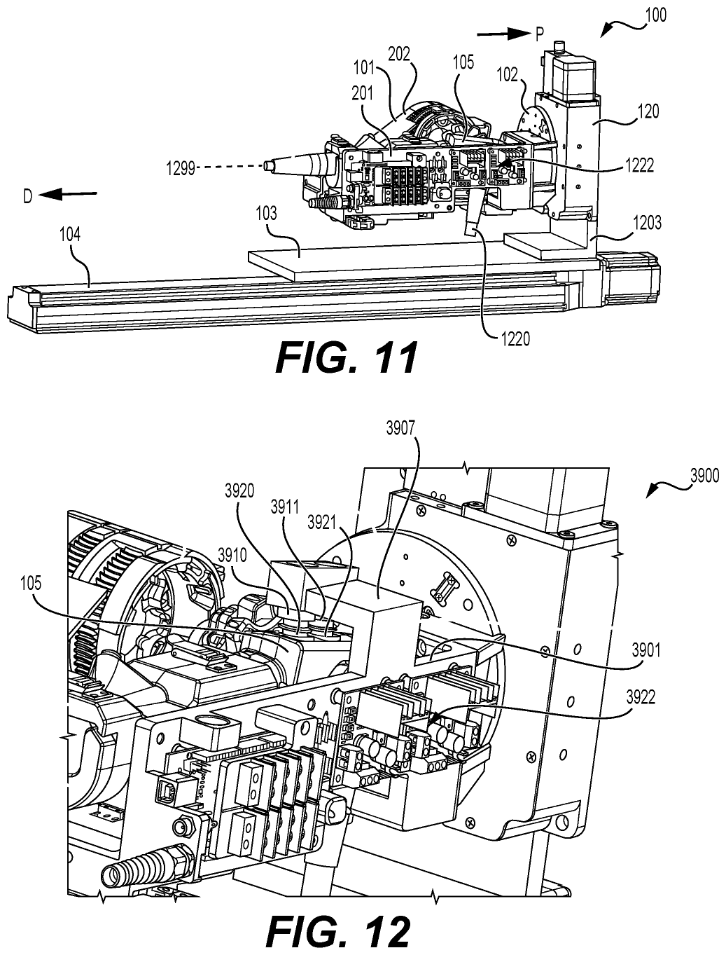

Cradle assembly 150 may be positioned between first body 201 and second body 202, within a recessed portion/channel 250 of first body 201. Recessed portion/channel 250 may be configured to receive handle assembly 140, and may include an opening 269 (FIG. 2) at a distal end of recessed portion/channel 250. First body 201 may include one or more ports 263, 264 for connection to control unit 199, a power supply, a USB device, or other device. A base portion 205 at a proximal end of first body 201 may be configured to couple rotary drive 102. A pair of rail portions 219, 220 may extend outward from first body 201 and may movably couple first body 201 to second body 202. One or more electronic circuit boards, a power supply such as a battery, and other electronic components known in the art may be housed within first body 201 (See circuitry 1222 in FIG. 11). Alternatively, one or more of the one or more electronic circuit boards, the power supply (e.g., the battery), and other electronic components may be remote from first body 201, with one or more wires being routed from the one or more remote components to first body 201. A locking button 296 may control the lock and release of first body 201 and second body 202, which will be discussed in further detail herein below.

Second body 202 may be moveably coupled to first body 201, such that a gear assembly 229 of second body 202 may move towards or away from first body 201. Second body 202 may be moveably coupled to first body 201 via one or more rail portions 219, 220, and gear assembly 229 may be moveable towards and away from channel 250 of first body 201. A distalmost portion 216 of second body 202 may be aligned with distal portion 217 of first body 201. The proximal end 275 of second body 202 may be distal from base portion 205 of first body 201, and a gap 276 may extend between proximal end 275 and base portion 205. Gap 276 may be configured to receive an umbilicus of endoscope 105, and may allow a user to position endoscope 105 within channel 250 without disconnecting endoscope 105 from control unit 199 and/or without removing a shaft of endoscope 105 from a patient. A cover 277 of second body 202 may be tapered such that a height of second body 202 decreases from a proximal end of cover 277 to a distal end of cover 277.

Rail portions 219, 220 of first body 201 may be retractable inward into first body 201 and extendable outward away from first body 201. In some examples, rail portions 219, 220 may be retractable and extendable via one or more electronic motors positioned within first body 201. In other examples, rail portions 219, 220 may be retractable and extendable via a user manually pushing or pulling second body 202 and/or first body 201 to extend or retract rail portions 219, 220. In some examples, rail portions 219, 220 may be locked in a position via actuation of locking button 296, and may be released via a subsequent actuation of locking button 296. Locking button 296 allows a user to lock control assembly 101 in an open or closed configuration. When an actuation of locking button 296 releases the lock of rail portions 219, 220, second body 202 may be free to slide towards and away from first body 201 via the extension and retraction of rail portions 219, 220.

FIG. 4 shows a perspective view of second body 202 with cover 277 removed to expose interior portions of second body 202. Lateral extensions 291, 292 of second body 202 may couple to rail portions 219, 220 of first body 201 via couplers 410, 411. Gear assembly 229 may include a first gear 403 and a second gear 404. First gear 403 may be adjacent to second gear 404, and each of first gear 403 and second gear 404 may rotate about an axis 499. A circular portion 407 of second gear 404 may help to restrict movement of first gear 403 and/or second gear 404, and may only allow first gear 403 and/or second gear 404 to rotate about axis 499. Circular portion 407 may be positioned at a proximal portion of second body 202. A first worm gear 405 may abut or be proximate to first gear 403 and engage with first gear 403, and a second worm gear 406 may abut or be proximate to second gear 404 and engage with second gear 404. Each of first worm gear 405 and second worm gear 406 may extend longitudinally from gear assembly 229 to a first motor 401 and a second motor 402, respectively. First motor 401 and second motor 402 may each be positioned at a distal portion of second body 202, and may be adjacent to each other. First motor 401, second motor 402, and any other motors discussed herein may be high precision motors, such as stepper motors or servo motors fitted with position encoders. The operation of first motor 401 and second motor 402 will be discussed in further detail with relation to FIG. 6 herein below.

First gear 403 may include a first lumen 235 extending though first gear 403 along axis 499, and second gear 404 may include a second lumen 236 extending though second gear 404 along axis 499. First lumen 235 and second lumen 236 may be connected to form a continuous passage through first gear 403 and second gear 404, including circular portion 407. Second lumen 236 may extend from first lumen 235 to an opening 230 of circular portion 407. First lumen 235 may have a smaller diameter than second lumen 236, and first lumen 235 may be configured to receive knob 171 of endoscope 105. Second lumen 236 may be configured to receive knob 170 of endoscope 105. A third lumen 237 may extend through at least a portion of first gear 403. Third lumen 237 may be connected to second lumen 236 at an opposite side from first lumen 235, and third lumen 237 may have a smaller diameter than second lumen 236. Third lumen 237 may also have a smaller diameter than first lumen 235. Third lumen 237 may be configured to receive a locking knob 172 of endoscope 105. In some examples, any of first lumen 235, second lumen 236, and/or third lumen 237 may be a recess in first gear 403 or second gear 404, including circular portion 407.

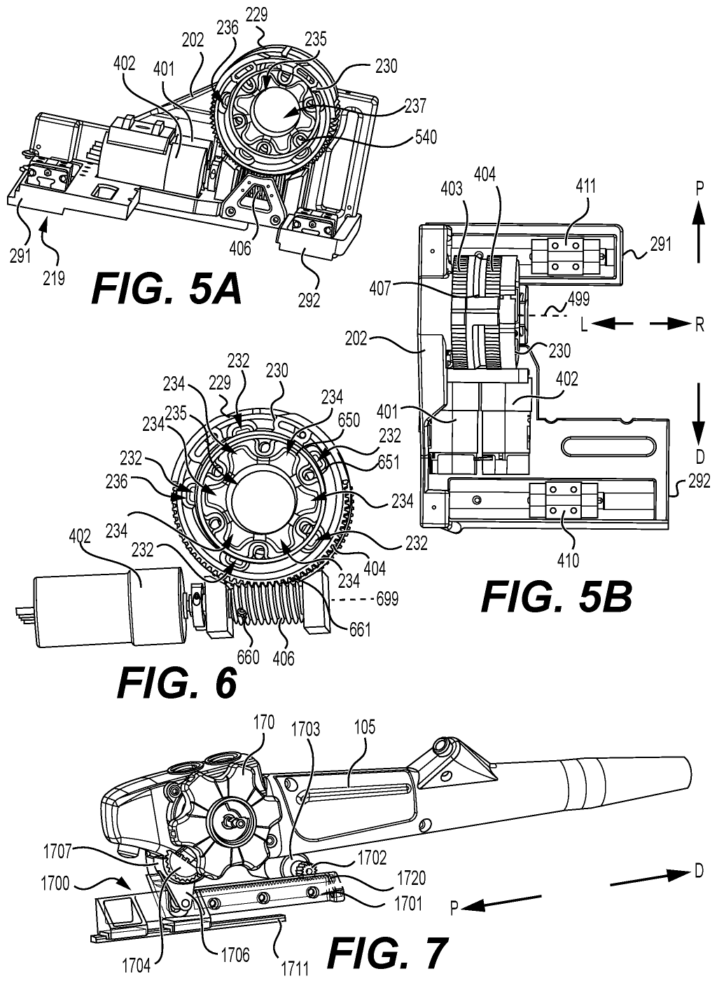

FIGS. 5A and 5B illustrate a side view and a top view, respectively, of second body 202 with cover 277 removed to expose interior portions of second body 202. First motor 401 may rotate first worm gear 405 about its longitudinal axis, which may then rotate first gear 403 about axis 499. Second motor 402 may rotate second worm gear 406 about its longitudinal axis, which may then rotate second gear 404 about axis 499. As shown in FIG. 5B, second body 202 may be moveable in a right or left direction, shown by R arrow and L arrow in FIG. 5B, relative to first body 201 to position gear assembly 229 over knobs 170, 171 of endoscope 105. A detector system 540 may be incorporated into gear assembly 229, and may be configured to detect the position of knobs 170, 171 of endoscope 105. Detector system 540 may include one or more cameras and/or one or more sensors. Detector system 540 will be discussed in further detail below.

FIG. 6 illustrates gear assembly 229, second motor 402, and second worm gear 406 removed from second body 202. As shown in FIG. 6, first lumen 235 may be at least partially formed by a series of recesses 234 circumferentially spaced around a radially-inward facing surface 650, relative to axis 499, of first gear 403. Each recess 234 may be configured to receive a portion of knob 171, such as a prong of knob 171. Recesses 234 of first gear 403 may allow knob 171 to temporarily couple to first gear 403 when first gear 403 is positioned in recesses 234 such that rotation of first gear 403 results in rotation of knob 171 about axis 499.

Second lumen 236 may be at least partially formed by a series of recesses 232 circumferentially spaced around a radially-inward facing surface 651, relative to axis 499, of second gear 404. Each recess 232 may be configured to receive a portion of knob 170, such as a prong of knob 170. Recesses 232 of second gear 404 may allow knob 170 to temporarily couple to second gear 404 when second gear 404 is positioned in recesses 232 such that rotation of second gear 404 results in rotation of knob 170 about axis 499.

A helical protrusion 660 of second worm gear 406, which may wind around a central longitudinal axis 699 of second worm gear 406, may abut gear teeth 661 of second gear 404, as shown in FIG. 6. When second motor 402 rotates second worm gear 406, helical protrusion 660 pushes gear teeth 661 in the proximal or distal direction, which rotates second gear 404 about axis 499. When knob 170 of endoscope 105 is positioned within recesses 232, the rotation of second gear 404 causes knob 170 to rotate, clockwise or counter-clockwise, about axis 499 (FIG. 5B). Since power cannot be transmitted from second gear 404 to worm gear 406, worm gear 406 provides a “self-locking” mechanism to lock knob 170 in place when second motor 402 is not driving (e.g., not rotating second worm gear 406). This “self-locking” mechanism provides control system 100 with a mechanism to lock a position of an articulated tip section of endoscope 105 in place, without the need for the use of any locking levers of endoscope 105. First motor 401, first worm gear 405, and first gear 403 operate in the same manner as second motor 402, second worm gear 406, and second gear 404 to rotate and/or lock knob 171 when knob 171 is positioned within recesses 234. Although worm gears 405, 406 are discussed herein, other gearing mechanisms may be used to drive gears 403, 404, such as conventional gearing assemblies, spur gears, helical gears, miter gears, or screw gears.

FIG. 7 illustrates endoscope 105 with an actuator assembly 1700 that may be incorporated into control system 100 to actuate an elevator lever 1704 of endoscope 105. Actuator assembly 1700 may be incorporated into first body 201 or second body 202, and may be positioned within channel 250. Actuator assembly 1700 may include a rack 1701, an elevator gear 1702, an elevator motor 1703, a frame 1711, and an elevator actuator 1706 rotatably coupled to a proximal end of rack 1701. The rack 1701 may be moveably mounted to frame 1711 such that rack 1701 may move only in a proximal and distal direction along a longitudinal axis of rack 1701. Teeth 1720 may extend longitudinally along an exterior surface of rack 1701, and teeth 1720 may engage elevator gear 1702. Elevator actuator 1706 may include a recessed portion 1707 configured to receive a portion of an elevator lever 1704 of endoscope 105. When elevator motor 1703 rotates elevator gear 1702, rack 1701 may translate in a proximal or distal direction to move elevator actuator 1706 proximally or distally, thus moving elevator lever 1704 proximally or distally to actuate an elevator of endoscope 105. When elevator motor 1703 stops driving elevator gear 1702, the elevator of endoscope 105 may be locked in its position. Elevator actuator 1706 may be spring-loaded and/or may be biased towards elevator lever 1704.

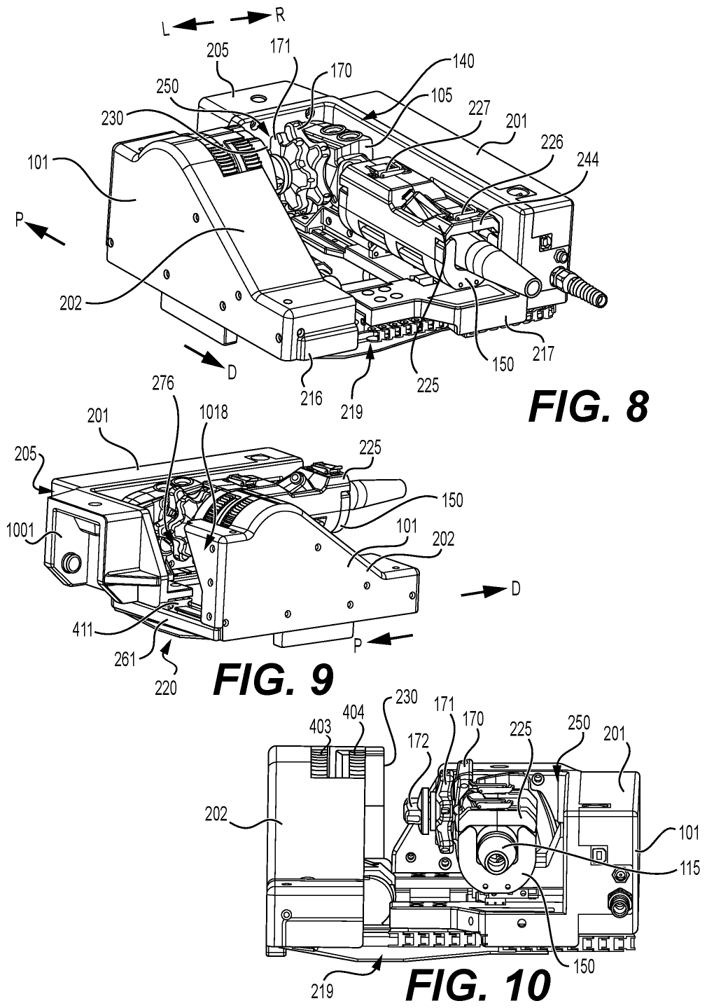

FIGS. 8, 9, and 10 illustrate perspective, rear, and front views, respectively, of control assembly 101 with endoscope 105 positioned within cradle assembly 150. Control assembly 101 is shown in FIGS. 8, 9, and 10 in an open configuration, with second body 202 spaced from endoscope 105 and cradle assembly 150. As shown in FIG. 8, opening 230 of second body 202 is laterally aligned with knobs 170, 171. Cradle assembly 150 may be positioned on first body 201 such that knobs 170, 171 are laterally aligned with opening 230 when endoscope 105 is positioned within cradle assembly 150. Second body 202 may move, via sliding along rail portions 219, 220, in a right or left direction (shown as L and R arrows in FIG. 8) relative to first body 201. FIG. 9 illustrates gap 276 between first body 201 and second body 202. As shown in FIG. 11, base portion 205 may include a recess 1001 configured to receive a portion of rotary drive 102 to fixedly couple base portion 205 to rotary drive 102. A proximalmost end 1018 of second body 202 is shown distal from base portion 205.

To position endoscope 105 in control system 100, a user may first position handle assembly 140 of endoscope 105 in cradle assembly 150, and temporarily fixedly couple handle assembly 140 to cradle assembly 150 by locking gate 225. Once handle assembly 140 is locked in cradle assembly 150, the user may transition control assembly 101 from an open configuration (shown in FIGS. 8-10) to a closed configuration (shown in FIG. 2). To move gear assembly 229 from the open configuration to the closed configuration, recesses 234 of first gear 403 need to be aligned with prongs of knob 171, and recesses 232 of second gear 404 need to be aligned with prongs of knob 170.

In some examples, detector system 540 may be used to automatically aligned first gear 403 with knob 171 and second gear 404 with knob 170. For example, control unit 199 may receive images of knobs 170, 171 from one or more cameras of detector system 540, and detector system 540 and/or control unit 199 may detect the position of knobs 170, 171. In other examples, control unit 199 may receive data from one or more sensors of detector system 540, and detector system 540 and/or control unit 199 may detect the position of knobs 170, 171. Detector system 540 and/or control unit 199 may detect a series of fiducial markers on endoscope 105 for reference, for example the center of knobs 170, 171, the position of the handle of endoscope 105, and the position of a prong of each of knobs 170, 171. Detector system 540 and/or control unit 199 may use the detected fiducial markers to determine an offset angle for each of knobs 170, 171. Detector system 540 and/or control unit 199 may then activate motors 401, 402 to move gears 403, 404 into alignment with knobs 170, 171, to allow control assembly 101 to transition from an open configuration to a closed configuration in which knobs 170, 171 are received within gear assembly 229. In other examples, a user may manually rotate first gear 403 and second gear 404 to align gears 403, 404 with the position of knobs 170, 171.

In some examples, detector system 540 and/or control unit 199 may detect the position of one or more locking knobs 172 and/or locking levers, and may provide a warning to the user if a locking knob 172 or locking lever is in a locked position (or has not been disabled). Control system 100 may provide this warning to the user via an audible alert, a visual display on an electronic display of control unit 199 or other electronic device, and/or via haptic feedback, such as vibration of a portion of control system 100 or other device. In some examples, control assembly 101 may include motors and/or gears to automatically move locking knob 172 and/or a locking lever from a locked position to an unlocked position.

In some examples, detector system 540 and/or control unit 199 may detect the position of elevator lever 1704. For example, camera system and/or control unit 199 may use fiducial markers on elevator lever 1704 and/or the handle of endoscope 105. Detector system 540 and/or control unit 199 may automatically align elevator actuator 1706 with elevator lever 1704 by actuating elevator motor 1703 and moving rack 1701 such that elevator actuator 1706 is aligned with elevator lever 1704.

FIG. 11 illustrates control system 100 coupled to endoscope 105, with a portion of first body 201 removed to expose electronic circuitry 1222. Electronic circuitry 1222 may be positioned within first body 201. A portion of an umbilicus 1220 of endoscope 105 is shown positioned within gap 276. A mount 1203 may be coupled to base plate 103 to couple rotary drive 102 to base plate 103. One or more motors may be incorporated into rail assembly 104 to move base plate 103, along with rotary drive 102 and control assembly 101, relative to rail assembly 104. Rail assembly 104 provides a mechanism to translate endoscope 105 proximally or distally, to move a shaft of endoscope 105 proximally or distally through a patient. In some examples, rail assembly 104 may include a scale or other demarcations to indicate the distance endoscope 105 has moved along rail assembly 104. In some examples, the distance traveled by endoscope 105 along rail assembly 104 may be displayed on an electronic display, such as an electronic display of control unit 199. Rotary drive 102 may rotate about axis 1299 to rotate endoscope 105 about a longitudinal axis of endoscope 105. In some examples, control system 100 may be connected to a control unit 199, and control unit 199 may control the operation of control system 100, such as the movement of endoscope via control system 100. In some examples, a remote control may be wirelessly connected or connected via a wire to control system 100 and/or control unit 199. Using the remote control, a user may actuate control system 100 to move endoscope 105 proximally or distally, rotate endoscope about axis 1299, actuate knobs 170, 171 to move a distal articulation portion of endoscope, and/or actuate the movement of an elevator of endoscope 105. The remote control may be similar to remote control units known in the art, such as a Playstation® style remote control or other handheld remote control unit.

FIG. 12 illustrates an alternative embodiment of a control system 2900 that may have any of the features discussed herein in relation to control system 100. Control system 3900 includes an actuator assembly 3907 including a first actuator 3910 and a second actuator 3911. Actuator assembly 3907 may be L-shaped and may extend outward from a first body 3901 of control system 3900. First body 3911 may have any of the features described herein in relation to first body 201. In some examples, actuator assembly 3907 may extend from a circuit board of circuitry 3922. Actuator assembly 3907 may include a first actuator 3910 and a second actuator 3911. When endoscope 105 is coupled to control system 3900, first actuator 3910 may be configured to align with and engage with a button actuator 3920 of endoscope 105, and second actuator 3911 may be configured to align with and engage with a button actuator 3921 of endoscope 105. For example, first actuator 3910 and second actuator 3911 may be moveable to control actuators of endoscope 105. Button actuators 3920, 3921 may be air/water actuators, suction actuators, imaging actuators, and/or may control any other aspect of endoscope 105. First actuator 3910 and second actuator 3911 may provide control system 3900 with the ability to control actuation of deployment or delivery of air/water from endoscope 105, application of suction at a target site using endoscope 105, initiating imaging systems, such as taking a picture or video using a camera of endoscope 105, and/or control other aspects of endoscope 105.

A shaft of endoscope 105 is designed to be flexible to maneuver through tortuous passages of a body. When a shaft of endoscope 105 is not supported, the shaft may develop a loop when control system 100, 3900 advances endoscope 105 distally or proximally, for example through movement of base plate 103 relative to rail assembly 104. Such a loop in the shaft may prevent endoscope 105 from being inserted into a patient or may prevent the full extent of movement of base plate 103 (and control assembly 101, rotary drive 102, base assembly 120, etc.) from being transmitted to a distal tip of the shaft. In order to prevent this undesirable looping, a support mechanism may be used with control system 100, 2900.

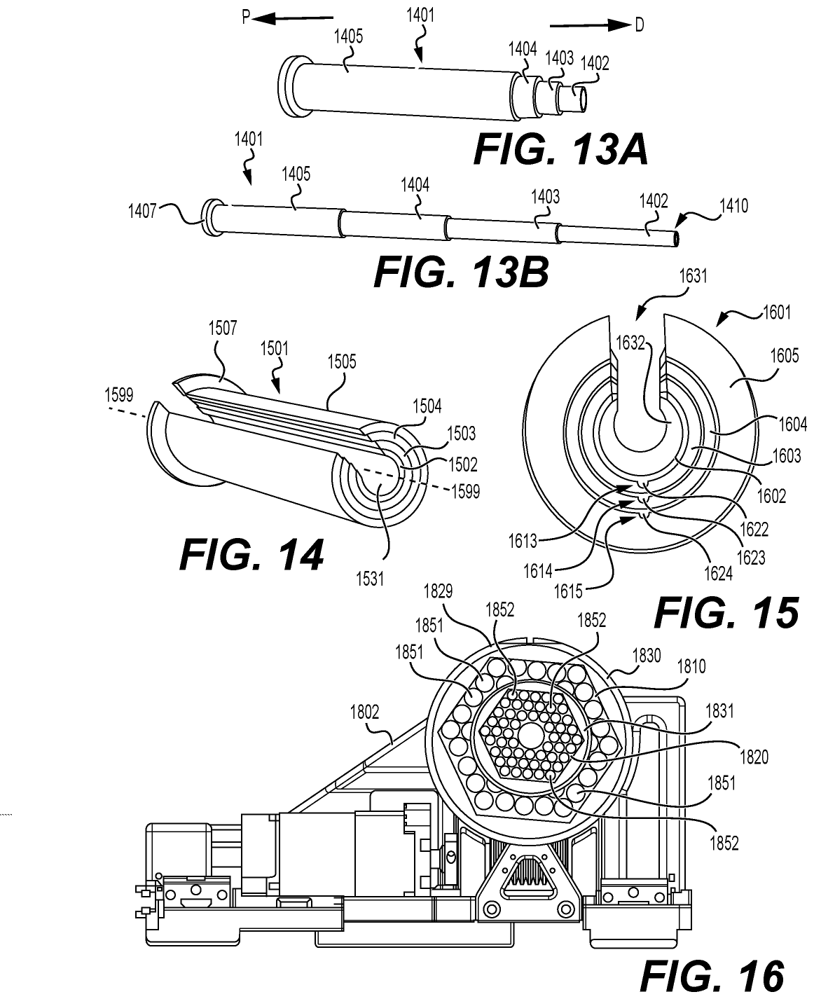

FIGS. 13A and 13B illustrate a telescopic support assembly 1401 including a series of concentric portions 1402-1405. Telescopic support assembly 1401 may transition from a retracted configuration shown in FIG. 13A to an extended configuration shown in FIG. 13B. Telescopic support assembly 1401 may be adjustable to adjust the longitudinal length of the assembly. Each portion 1402-1405 may be tubular and a lumen may extend longitudinally through each portion 1402-1405. Portion 1402 may be received within the lumen of portion 1403. Portion 1403 may be received within a lumen of portion 1404, and portion 1404 may be received within a lumen of portion 1405. In some examples, each portion 1402-1405 may include a circumference that tapers along the portion's longitudinal length such that its circumference at a proximal end is larger than a circumference at its distal end. The tapering of each portion 1402-1405 may facilitate retaining the smaller portion 1402-1405 received within the larger portion 1402-1405, even when each portion 1402-1405 is extended distally. A proximal end 1407 of telescopic support assembly 1401 may be fixedly coupled to a distal end of cradle assembly 150, and proximal end 1407 may be wider than a distal portion of support assembly 1401 (e.g., to be retained within the distal end of cradle assembly 150). Each portion 1402-1405 may be configured to receive a portion of a shaft of endoscope 105 within its respective lumen. A distal end 1410 of telescopic support assembly 1401 may be coupled to a fixed point during operation, such as the rail assembly 104 or to a bed supporting a patient. Telescopic support assembly 1401 may extend or contract in length as endoscope 105 is moved proximally or distally, via movement of cradle assembly 150 of control system 100, 2900, during a procedure. In some examples, telescopic support assembly 1401 may be positioned around a shaft of endoscope 105 prior to inserting the shaft into a patient.

FIG. 14 illustrates an alternative embodiment of a telescopic support assembly 1501, which may have any of the features described herein in relation to telescopic support assembly 1401. Telescopic support assembly 1501 includes concentric portions 1502-1505, and each portion 1502-1505 includes a slot 1531 extending longitudinally across the entire length of the portion 1502-1505. A central longitudinal axis 1599 of telescopic support assembly 1501 may extending through slot 1531. Note telescopic support assembly 1501 is shown in a collapsed configuration with slot 1531 consisting of the slot 1531 of portion 1502, however, when telescopic support assembly 1501 is in a fully expanded configuration, slot 1531 includes slots from each of portions 1502-1505. Slot 1531 is configured to receive a shaft of endoscope 105 such that a user may slide a proximal portion of the shaft of endoscope 105 into slot 1531 intraoperatively, without removing the shaft from a patient. A proximal end 1507 of telescopic support assembly 1501 may be fixedly coupled to a distal end of cradle assembly 150, and proximal end 1507 may be wider than a distal portion of support assembly 1501 (e.g., to be retained within the distal end of cradle assembly 150).

FIG. 15 illustrates an alternative embodiment of a telescopic support assembly 1601, which may have any of the features described herein in relation to telescopic support assemblies 1401, 1501. Telescopic support assembly 1601 may include concentric portions 1602-1605 and a longitudinal slot 1631 extending the entire length of telescopic support assembly 1601. A radially-inward facing surface 1632 of portion 1602 may form a portion of longitudinal slot 1631. Each portion 1602, 1603, 1604 may include a radial protrusion 1622, 1623, 1624, respectively, extending longitudinally along the length of the portion 1602, 1603, 1604. Each radial protrusion 1622, 1623, 1624 may be located, in some examples, along a central region of the portion 1602, 1603, 1604. Each portion 1603, 1604, 1605 may include a longitudinal recess 1613, 1614, 1615, respectively, extending longitudinally along the length of the portion 1603, 1604, 1605. Each longitudinal recess 1613, 1614, 1615 may be configured to receive a radial protrusion 1622, 1623, 1624 of an adjacent portion 1602-1604. By incorporating radial protrusions 1622-1624 and longitudinal recesses 1613-1615 in telescopic support assembly 1601, each portion 1602-1605 may be prevented from rotating relative to other portions and slot 1631 may be maintained during expansion and retraction of telescopic support assembly 1601.

FIG. 16 illustrates an alternative embodiment of a second body 1802 that may be incorporated into any of control systems 100, 2900 discussed herein, for example in place of second body 202. Second body 1802 may have any of the features described herein in relation to second body 202, and includes an opening 1830 of gear assembly 1829. A first recess 1810 of a first gear of gear assembly 1829 may include a series of spring-biased pins 1851 within first recess 1810. First recess 1810 may be configured to receive knob 170 of endoscope 105, and some of spring-biased pins 1851 may compress when knob 170 is positioned within recess 1810. Spring-biased pins 1851 may conform to the shape of knob 170 such that, when knob 170 is received within first recess 1810, knob 170 rotates when the first gear rotates. The depth of recess 1810 may be substantially equal to the width of knob 170, and each spring-biased pin 1851 may be biased towards an extended position in which the spring-biased pin 1851 extends the entire width of recess 1810.

A second recess 1820 of a second gear 1831 of gear assembly 1829 may include a series of spring-biased pins 1852 within second recess 1820. Second recess 1820 may be configured to receive knob 171 of endoscope 105, and some of spring-biased pins 1852 may compress when knob 171 is positioned within second recess 1820. Spring-biased pins 1852 may conform to the shape of knob 171 such that, when knob 171 is received within second recess 1820, knob 171 rotates when the second gear 1831 rotates. The depth of recess 1820 may be substantially equal to the width of knob 171, and each spring-biased pin 1852 may be biased towards an extended position in which the spring-biased pin 1852 extends the entire width of recess 1820.

The gear assembly 1829 of FIG. 16 may facilitate the positioning of knobs 170, 171 within gear assembly 1829. By incorporating gear assembly 1829 into a control system 100, 2900, a user may not have to align any specific recesses/lumens of gears of gear assembly 1829 before inserting knobs 170, 171 into gear assembly 1829. For example, after a user couples handle assembly 140 to cradle assembly 150, a user may simple slide second body 1802 towards handle assembly 140 to insert knobs 170, 171 into gear assembly 1829. Spring biased pins 1851, 1852 may then conform to the shape of knobs 170, 171, and the user may proceed with operation of control system 100, 2900. This may reduce total procedure time and facilitate operation of control system 100, 2900. The hexagonal arrangement of spring biased pins 1851, 1852 may help to ensure that spring biased pins 1851, 1852 surround knobs 170, 171. In other examples, the arrangement of spring biased pins 1851, 1852 may be other shapes, such as triangular, rectangular, etc.

To operate control system 100 or 2900, a user may first insert handle assembly 140 of endoscope 105 into cradle assembly 150. The user may close and lock gate 225 using fasteners 226, 227 to couple handle assembly 140 to cradle assembly 150. In some examples, control system 100 may then automatically align knobs 170, 171 with recesses 232, 234 of gear assembly 229 using detector system 540 and/or control unit 199. In other examples, the user may manually rotate gears 403, 404 of gear assembly 229 to align recesses 232, 234 with knobs 170, 171. The user may then slide second body 202 towards cradle assembly 150 to position knobs 170, 171 in gear assembly 229, and lock second body 202 in a closed position. In some examples, a user may then couple a telescopic support assembly 1401, 1501, 1601 to cradle assembly 150 and position the telescopic support assembly 1401, 1501 over a portion of a shaft of endoscope 105. In some examples, a user may couple telescopic support assembly 1501, 1601 to cradle assembly 150 and position telescopic support assembly 1501, 1601 over a portion of a shaft of endoscope 105 after the shaft has been inserted into a patient, such as by inserting the shaft into slots 1531, 1631.

A user may then proceed to manipulate endoscope 105 using control system 100, 2900 to conduct a procedure. The user may utilize control unit 199 to actuate movement of an articulation portion of endoscope 105 via rotation of knobs 170, 171; may rotate endoscope 105 via driving rotary drive 102, and may translate endoscope 105 proximally or distally by actuating a motor within rail assembly 104 to move base plate proximally or distally. In some examples, the user may actuate an elevator actuator 1706 of endoscope 105 to move an elevator at a distal portion of endoscope 105. Endoscope 105 may include an internal lumen, and moving the elevator may direct a medical device delivered to the treatment site through the internal lumen. In some examples, a user may use a remote control, such as a handheld controller (e.g., with one or more joysticks), a laptop, tablet, cellphone, or other device, to actuate any of these operations of control system 100, 2900. During a procedure, a user may remove endoscope 105 from control assembly 101 to manually operate endoscope 105, and in some examples, may then re-insert endoscope 105 into control assembly 101 to operate endoscope 105 using control system 100.

In some examples, control system 100, 2900 may include one or more actuators to perform one or more predetermined medical device movements. For example, a user may select a “home” button on a user interface of control unit 199 or a button on a remote control, and control system 100, 2900 may execute one or more movements/actuations to move an articulation section of a shaft of endoscope 105 to a straight position, or a position in which the articulation section is longitudinally aligned with a proximal portion of the shaft of endoscope 105. This may facilitate withdrawal of endoscope 105 from a patient. In other examples, control system 100, 2900 may include one or more actuators to perform one or more predetermined medical device movements to achieve specific positions of the articulation section of endoscope 105, such as a 45 degree rightward bend, a 45 degree leftward bend, a 90 degree upward bend, a 45 degree downward bend, or a 180 degree bend. This may allow a user to simply press a button to have control system 100, 2900 move endoscope 105 into a specific position. Various predetermined movements could be pre-set (e.g., factory stored movements) or may be user-stored/saved movements (e.g., a user's “favorites”). In some examples, control system 100, 2900 may include a remote image capture switch, such as a foot pedal or a lever, to capture an image using endoscope 105.

In some examples, control system 100, 2900 may be designed to allow a user to input a depth of endoscope insertion into the patient into an electronic interface of control unit 199, and control system 100, 2900 may automatically move endoscope 105 the input insertion distance. In some examples, to facilitate insertion of endoscope 105 into control system 100, 2900 (e.g., when transitioning from manual operation to robotic operation), control system 100, 2900 can also be designed to allow the user to input the current depth of endoscope 105 within a patient intraoperatively, and control system 100, 2900 will then automatically move control assembly 101 to an appropriate position for insertion of handle assembly 140 into control assembly 101 (e.g., to compensate for the depth of the endoscope insertion).

In various embodiments, any of the systems and methods described herein may include control unit 199, control system 100, 2900, and a medical device (e.g., endoscope 105). Control unit 199 and/or control system 100, 2900 may include a processor, in the form of one or more processors or central processing unit (“CPU”), for executing program instructions. In some examples, the one or more processors may be one or more processing boards. Control unit 199 and/or control system 100, 2900 may include an internal communication bus, and a storage unit (such as ROM, HDD, SDD, etc.) that may store data on a computer readable medium, although control unit 199 and/or control system 100, 2900 may receive programming and data via network communications. Control unit 199 and/or control system 100, 2900 may also have a memory (such as RAM) storing instructions for executing techniques presented herein, although the instructions may be stored temporarily or permanently within other modules of control unit 199 and/or control system 100, 2900 (e.g., processor and/or computer readable medium) or remotely, such as on a cloud server electronically connected with control unit 199 and/or control system 100, 2900. The various system functions of control unit 199 and/or control system 100, 2900 may be implemented in a distributed fashion on a number of similar platforms, to distribute the processing load. Alternatively, the systems discussed herein may be implemented by appropriate programming of one computer hardware platform at control unit 199.

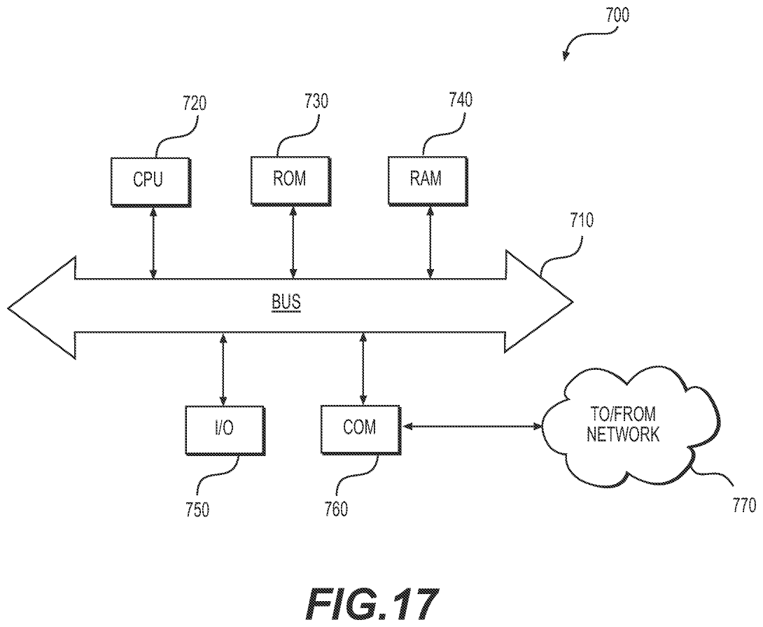

FIG. 17 provides a functional block diagram illustration of general purpose computer hardware platforms. FIG. 17 illustrates a network or host computer platform 700, as may typically be used to implement a server or a browser, or any other device executing features of the methods and systems described herein. It is believed that those skilled in the art are familiar with the structure, programming, and general operation of such computer equipment and as a result, the drawings should be self-explanatory.

A platform for a server or the like 700, for example, may include a data communication interface for packet data communication 760. The platform may also include a central processing unit (CPU) 720, in the form of one or more processors, for executing program instructions. The platform typically includes an internal communication bus 710, program storage, and data storage for various data files to be processed and/or communicated by the platform such as ROM 730 and RAM 740, although the platform 700 for the server often receives programming and data via network communications 770. The hardware elements, operating systems, and programming languages of such equipment are conventional in nature, and it is presumed that those skilled in the art are adequately familiar therewith. The platform 700 for the server also may include input and output ports 750 to connect with input and output devices such as keyboards, mice, touchscreens, monitors, displays, etc. Of course, the various server functions may be implemented in a distributed fashion on a number of similar platforms, to distribute the processing load. Alternatively, the servers may be implemented by appropriate programming of one computer hardware platform.

Program aspects of the technology discussed herein may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may, at times, be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

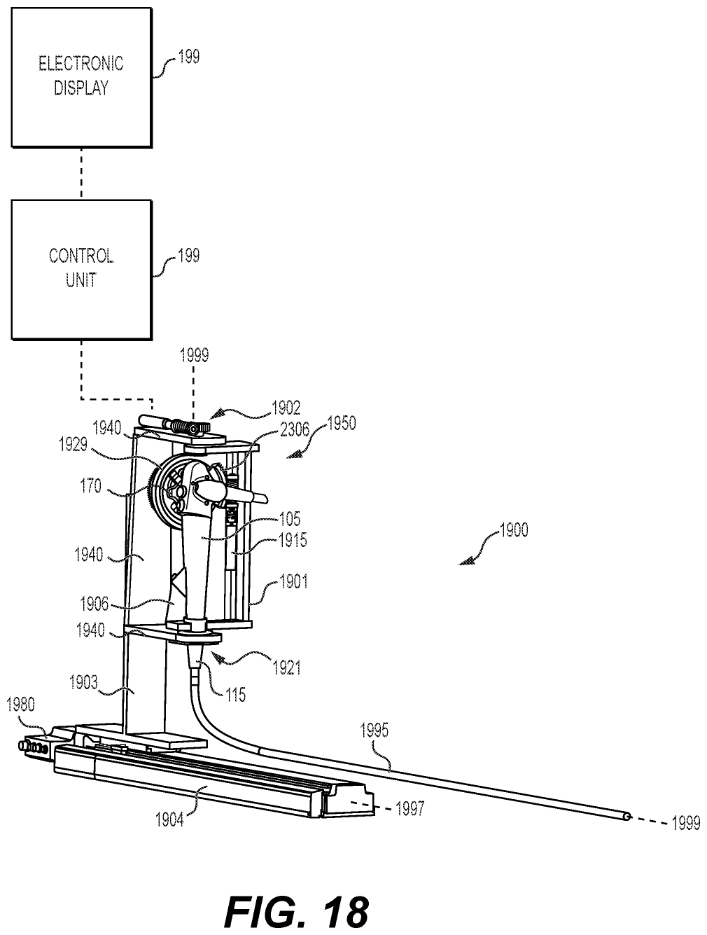

FIG. 18 illustrates an exemplary motorized control system 1900 in accordance with one or more embodiments of this disclosure. Motorized control system 1900 may include any of the features discussed herein in relation to motorized control system 100. Motorized control system 1900 may be configured to couple to a medical device, such as endoscope 105, via cradle assembly 1950. Cradle assembly 1950 may include a control assembly 1929, and a first frame 1901 may at least partially surround control assembly 1929. Cradle assembly 1950 may be coupled at its proximal end to a rotary drive 1902 and a base frame 1940, and may be connected to a control unit 199 (via one or more wires, wirelessly, or any other means of electrical connection). Rotary drive 1902 and base frame 1940 may be coupled to a support structure 1903, and base frame 1940 may be moveably coupled to support structure 1903. In some examples, a piece of base frame 1940 may include distal lumen 1921 and may be detachable to allow an endoscope or other medical device to be mounted and dismounted mid-procedure. In some examples, the detachable piece of base frame 1940 may rotate relative to a bearing on base frame 1940.

Support structure 1903 may be moveably coupled to a rail assembly 1904, and a rail motor 1980 may be configured to move base frame 1940 longitudinally relative to rail assembly 1904. As will be discussed in detail herein below, control system 1900 may be configured to control movement of endoscope 105 in a proximal and distal direction, control rotation of endoscope 105 about a central longitudinal axis 1999 in both clockwise and counter-clockwise directions, control movement of a distal articulation section of a shaft 1995 of endoscope 105 via rotation of knobs 170, 171, control movement of an elevator of endoscope 105 via movement of one or more levers of endoscope 105, and/or control actuation of one or more suction lumens or water jets of endoscope 105.

As shown in FIG. 18, endoscope 105 may couple to cradle assembly 1950 via base frame 1940, first frame 1901, and control assembly 1929. A proximal portion of shaft 1995 may extend through a distal lumen 1921 of base frame 1940 and first frame 1901. Knobs 170, 171 of endoscope 105 may be received by control assembly 1929, and a lever of endoscope 105 may be received by a lever actuator 2306 of control assembly 1929. Control assembly 1929 may be fixedly coupled to a mounting plate 1906 of first frame 1901 (such as via a bracket 2104 or other coupling assembly), and mounting plate 1906 may be substantially planar and extend from a proximalmost end of first frame 1901 to a distalmost end of first frame 1901. Lever actuator 2306 may be driven by a lever motor 1915 mounted to first frame 1901. Rotary drive 1902 may be coupled to a proximal end of base frame 1940, and rotatory drive 1902 may be configured to rotate cradle assembly 1950, including control assembly 1929 and first frame 1901, about central longitudinal axis 1999. Endoscope 105 may be in a vertical position when coupled to cradle assembly 1950, with central longitudinal axis 1999 substantially perpendicular to a central longitudinal axis 1997 of rail assembly 1904. Since motorized control system 1900 is configured to receive endoscope 105 in a vertical position, a user may more easily transition from holding endoscope 105 in a vertical position and operating on a patient to coupling endoscope 105 to motorized control system 1900. The design of motorized control system 1900 may prevent a user from having to move endoscope 105 from a vertical position to a horizontal position in order to couple endoscope 105 to motorized control system 1900, and thus may decrease procedure time and reduce user error when coupling endoscope 105 to motorized control system 1900.

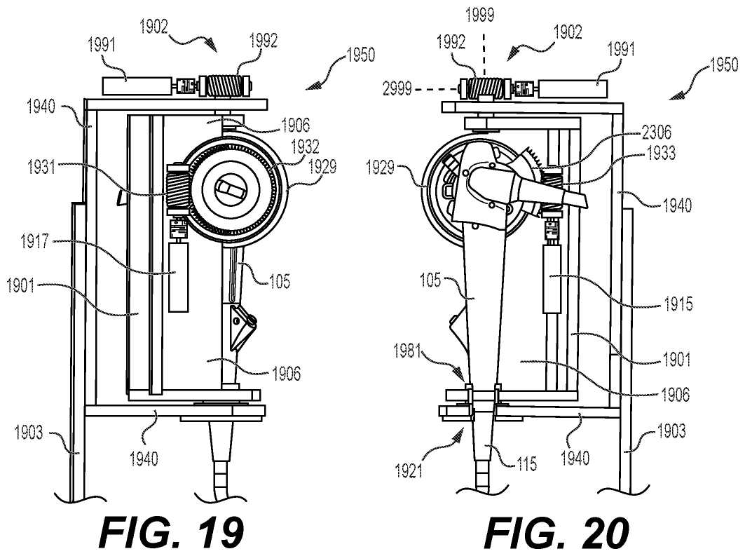

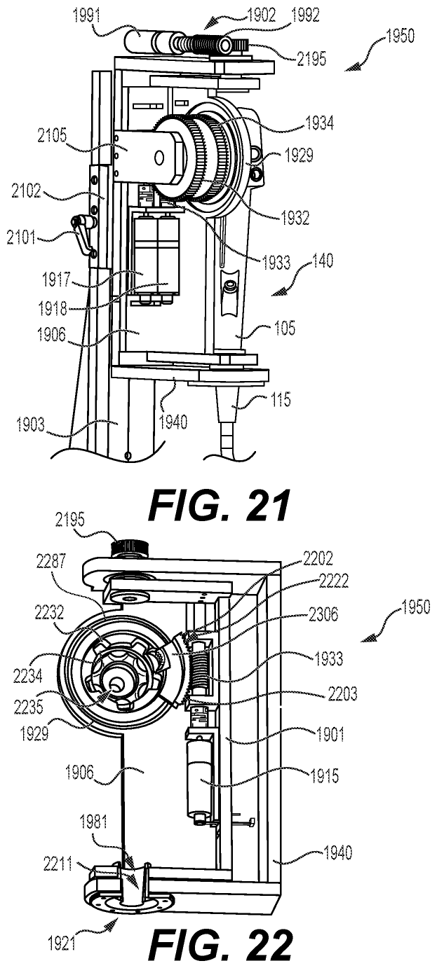

FIGS. 19 and 20 illustrate side views of cradle assembly 1950. As shown in FIG. 19, a first motor 1917 is coupled to mounting plate 1906 and first worm gear 1931. First motor 1917 is configured to rotate first worm gear 1931 about a central longitudinal axis of first worm gear 1931 to drive a first gear 1932 of control assembly 1929. Referring to FIG. 21, a second motor 1918 is also coupled to mounting plate 1906 and is configured to drive second worm gear 1933. Second motor 1918 is configured to rotate second worm gear 1933 about a central longitudinal axis of second worm gear 1933 to drive a second gear 1934 of control assembly 1929. Each of first motor 1917 and second motor 1918 extends longitudinally along a planar surface of mounting plate 1906. First worm gear 1932 may abut and engage with first gear 1932 and second worm gear 1933 may abut and engage with second gear 1934. As will be described in further detail below, first motor 1917, second motor 1918, first worm gear 1932, second worm gear 1933, first gear 1932 and second gear 1934 function in substantially the same manner as motors 401, 402 and worm gears 405, 406 described hereinabove. A lever 2101 may be coupled to support structure 1903 and a bracket 2102, and bracket 2102 may be fixedly coupled to base frame 1940. Lever 2101 may be configured to fixedly couple base frame 1940 to support structure 1903 in a first position, and moveably couple base frame 1940 to support structure 1903 in a second position so as to allow a user to slide base frame 1940 vertically up and down along support structure 1903. Lever 2101 may allow users to adjust the height of operation of endoscope 105 while using motorized control system 1900.

First frame 1901, along with mounting plate 1906 and control assembly 1929, may be rotatably coupled to rotary gear 2195 of rotary drive 1902. Rotary drive 1902 may rotate first frame 1901 about central longitudinal axis 1999 relative to base frame 1940 and support structure 1903. Distal lumen 1921 of base frame 1940 and distal lumen 1981 of first frame 1901 may be aligned with each other and distal lumens 1921, 1981 may be configured to receive endoscope 105, such as a distal end 115 of handle assembly 140. Each distal lumen 1921, 1981 may be tapered such that a distal opening of distal lumen 1921 is smaller than a proximal opening of distal lumen 1921, 1981. A longitudinal gap 2211 (shown in FIG. 22) may extending the entire longitudinal length of distal lumen 1981 and distal lumen 1921, and gap 2211 may be configured to receive shaft 1995 of endoscope 105. Distal lumens 1921, 1981 may removably couple to endoscope 105 and may facilitate holding endoscope 105 in a vertical position (e.g. the position shown in FIGS. 19 and 20). In some examples, distal lumen 1981 of first frame 1901 may be configured to removably, fixedly couple to handle assembly 140 and distal lumen 1921 may be configured to rotatably couple to handle assembly 140 so that handle assembly 140 may rotate within distal lumen 1921 about axis 1999 when positioned within distal lumen 1921. Longitudinal gap 2211 may allow a user to position endoscope 105 within distal lumens 1921, 1981 intraoperatively, while a proximal portion of shaft 1995 is positioned within a patient, and may avoid the need to remove shaft 1995 from the body during a procedure. Furthermore, longitudinal gap 2211 may provide a means for a user to couple endoscope 105 to cradle assembly 1950 without having to feed a distal end of shaft 1995 through lumens 2211, 1921, and may position shaft 1995 within lumens 2211, 1921 without feeding the distal end of shaft 1995 through lumens 2211, 1921.

Rotary drive 1902 may include a rotary motor 1991, a rotary worm gear 1992, and a rotary gear 2195. Each of rotary motor 1991, rotary worm gear 1992, and rotary gear 2195 may be positioned at a proximalmost end of base frame 1940, and may be positioned on a proximal-facing planar surface of base frame 1940. Rotary motor 1991 may drive rotary worm gear 1992 to rotate rotary worm gear 1992 about a central longitudinal axis 2999 (FIG. 20) of rotary worm gear 1992. Central longitudinal axis 2999 may be transverse from a central longitudinal axis of lever motor 1915. A helical groove of rotary worm gear 1992 may engage rotary gear 2195 to rotate rotary gear 2195 about axis 1999. Rotary gear 1995 may be fixedly coupled to first frame 1901 and may be rotatable coupled to base frame 1940. For example, rotary gear 2195 may rotate a shaft (note shown) extending from rotary gear 2195 through base frame 1940 to first frame 1901, and thus rotate control assembly 1929 and endoscope 105 about axis 1999. In some examples, first frame 1901 may be rotatably coupled to a proximal portion of base frame 1940, such as proximate to lumen 1921 of base frame 1940, and may be configured to allow first frame 1901 to be rotated about axis 1999.

As shown in FIGS. 19 and 20, endoscope 105 may couple to cradle assembly 1950 by positioning a distal end 115 of handle assembly 140 in distal lumens 1921, 1981, and positioning knobs 170, 171 in control assembly 1929. Knobs 170, 171 may removably couple to control assembly 1929, and lever actuator 2306 may couple to elevator lever 1704 of endoscope 105. In some examples, handle assembly 140 may be held in vertical position via control assembly 1929, distal lumens 1921, 1981, and lever actuator 2306.

FIG. 22 illustrates cradle assembly 1950 removed from motorized control system 1900. As shown in FIG. 22, lever actuator 2306 may include a curved radially-outer surface 2222 and teeth 2202 on the curved radially-outer surface 2222. Teeth 2202 may engage with worm gear 1933 such that worm gear 1933 may drive movement of lever actuator 2306 to actuate elevator lever 1704.

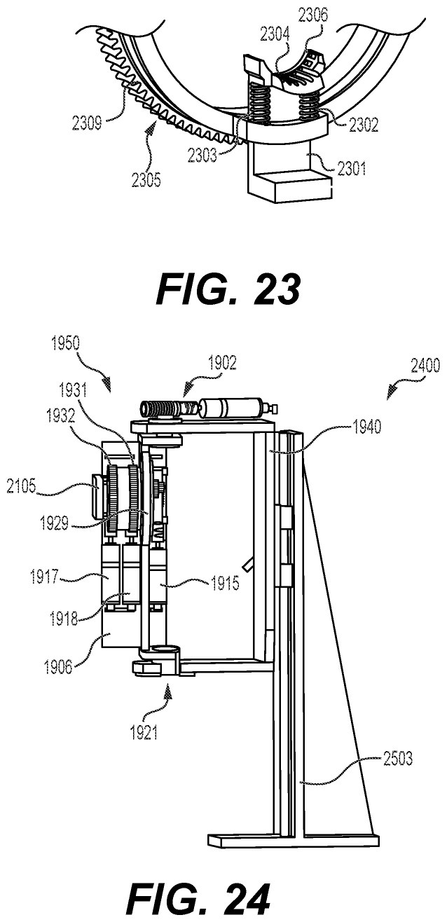

FIG. 23 illustrates an exemplary lever actuator 2306 including biasing members 2302, 2303, a curved radially-outer surface 2305 with teeth 2309 a curved radially-inward facing surface 2304 configured to abut elevator lever 1704, and a recessed portion 2301 configured to slideably receive a protruding portion 2287 of control assembly 1929 to moveably couple lever actuator 2306 to control assembly 1929. Biasing members 2302, 2303 may be springs and/or may be any biasing member known in the art, and in some examples lever actuator 2306 may include only a single biasing member 2302, 2303 or may include more than two biasing members 2303, 2303. Biasing members 2302, 2303 may provide a means for a user to move radially-inward facing surface 2304 and position elevator lever 1704 such that radially-inward facing surface 2304 abuts elevator lever 1704 once the user releases radially-inward facing surface 2304. Teeth 2309 may engage worm gear 1933. When lever motor 1915 rotates lever actuator 2306, 2306, lever actuator 2306, 2306 may move in a proximal or distal direction to move elevator actuator 1706 proximally or distally, thus moving elevator lever 1704 proximally or distally to actuate an elevator of endoscope 105. When lever motor 1915 stops driving lever actuator 2306, the elevator of endoscope 105 may be locked in its position.

Referring to FIG. 22, a first lumen 2235 may be at least partially formed by a series of recesses 2234 circumferentially spaced around a radially-inward facing surface 2232 of control assembly 1929. Control assembly 1929 may have any of the features of gear assembly 229, and control assembly 1929 may operate in substantially the same manner as gear assembly 229. Specifically, first motor 1917 may drive (rotate) first worm gear 1931 to rotate first gear 1932, and thus rotate knob 171 of endoscope 105. Second motor 1918 may drive (rotate) second worm gear 1933 to rotate second gear 1934, and thus rotate knob 170 of endoscope. Any of the features of gear assembly 229 may be incorporated into control assembly 1929. In some examples, a portion of control assembly 1929 may be integral with base plate 1906. First motor 1917 and second motor 1918 may be positioned on an opposite side of mounting plate 1906 as lever motor 1915.

In operation, motorized control system 1900 may be used in substantially the same manner as system 100 described hereinabove, and may incorporate any of the operational features discussed hereinabove in relation to system 100. A user may first position endoscope 105 at a target site within a body of a patient. The user may then couple handle assembly 140 to motorized control system 1900 by inserting knobs 170, 171 into control assembly 1929 and handle assembly 140 into distal lumens 1981, 1921. The user may then use a controller, such as a handheld controller, a computer, or other control unit, to actuate motors 1915, 1917, 1918 to move (articulate) endoscope 105 and move an elevator of endoscope 105 during the operation. The user may actuate rail motor 1980 to translate endoscope in a proximal or distal direction, and may actuate rotary drive 1902 to rotate endoscope 105 about axis 1999. Since motorized control system 1900 is configured to hold handle assembly 140 in a vertical positon, a user may more ergonomically switch between manual operation of endoscope 105 and motorized operation of endoscope 105 via motorized control system 1900, which may reduce user fatigue, increase procedure accuracy, reduce patient complications, and reduce overall surgical time, among other benefits.

FIG. 24 illustrates another embodiment of a motorized control system 2400. Motorized control system 2400 may include any of the features of any of the other systems, devices, and methods discussed herein, such as motorized control systems 100, 2900, 1900. Motorized control system 2400 may include cradle assembly 1950 with rotary drive 1902, gears 1931, 1932, motors 1915, 1917, 1918, mounting plate 1906, and base frame 1940. Support structure 2503 may be T-shaped or L-shaped, and may not be configured to move, comparted to support structure 1903 of motorized control system 1900. During operation of motorized control system 2400, a user may manually move shaft 1995 proximally or distally through a patient. Motorized control system 2400 may be configured to move endoscope 105 by rotating endoscope 105 about axis 1999, configured to control articulation of a distal portion of endoscope 105 via motorized control of knobs 170, 171, and may be configured to control actuation of an elevator of endoscope 105 via elevator lever 1704. By providing a fixed support structure 2503 in motorized control system 2400, the system 2400 may be fixed in a location within an operating room to facilitate operating room efficiency and reduce procedure time.

As discussed hereinabove, a shaft 1995 of endoscope 105 is designed to be flexible to maneuver through tortuous passages of a body. When a shaft of endoscope 105 is not supported, the shaft may develop a loop when control system 1900, 2600 or a user advances endoscope 105 distally or proximally. Such a loop in the shaft may prevent endoscope 105 from being inserted into a patient or tangle shaft 1995 and prevent movement of a proximal portion of shaft 1995 from being transmitted to a distal tip of the shaft 1995. In order to prevent this undesirable looping, a support mechanism may be used with control system 100, 2900, 1900, 2600.

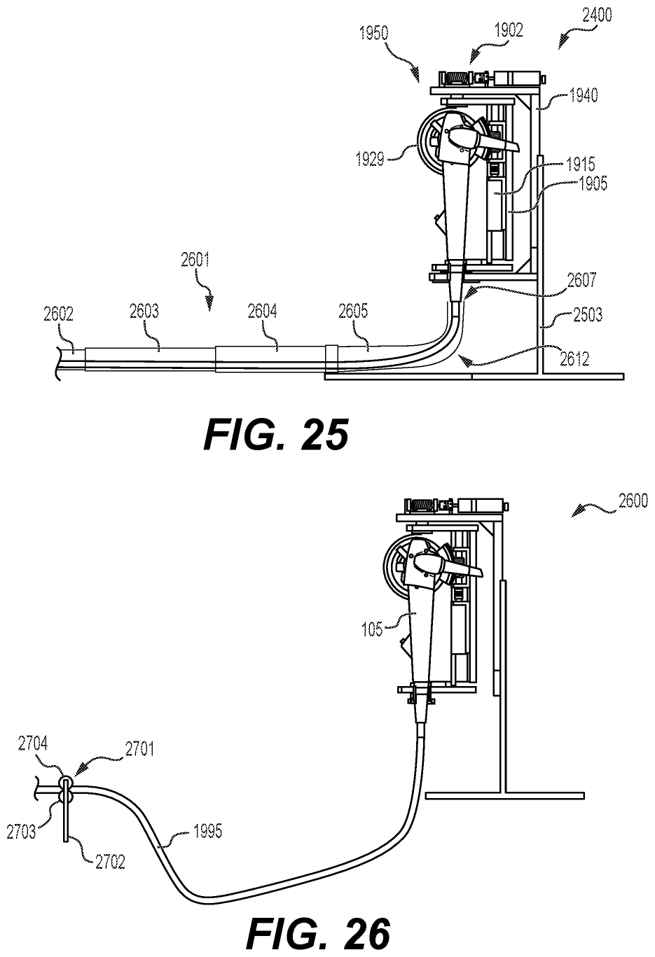

FIG. 25 illustrates a telescopic support assembly 2601 coupled to control system 2600. Telescopic support assembly 2601 may include any of the features discussed herein regarding telescopic support assemblies 1401, 1501. Telescopic support assembly 2601 may include a series of concentric portions 2602-2605. A proximalmost concentric portion 2605 may include a curved portion 2612 configured to support a curved proximal section of shaft 1995. A proximal opening 1607 of telescopic support assembly 1601 may face a direction transverse from a distalmost opening (not shown) of telescopic support assembly 2601. Telescopic support assembly 2601 may transition from a retracted configuration to an extended configuration shown in FIG. 25. Telescopic support assembly 2601 may be adjustable to adjust the longitudinal length of the assembly. Each portion 2602-2605 may be tubular and a lumen may extend longitudinally through each portion 2602-2605. Portion 2602 may be received within the lumen of portion 2603. Portion 2603 may be received within a lumen of portion 2604, and portion 2604 may be received within a lumen of portion 2605. In some examples, each portion 2602-2605 may include a circumference that tapers along the portion's longitudinal length such that its circumference at a proximal end is larger than a circumference at its distal end. The tapering of each portion 2602-2605 may facilitate retaining the smaller portion 2602-2604 received within the larger portion 2603-2605, even when each portion 2602-2605 is extended distally. In some examples, a proximal end of portion 2605 may be fixedly coupled to a distal end of cradle assembly 1950. Each portion 2602-2605 may be configured to receive a portion of a shaft 1995 of endoscope 105 within its respective lumen. A distal end of telescopic support assembly 2601 may be coupled to a fixed point during operation, such as the rail assembly 1904 or to a bed supporting a patient. Telescopic support assembly 2601 may extend or contract in length as endoscope 105 is moved proximally or distally, via movement of a control system 100, 2900, 1900, 2400 or via movement of endoscope 105 via a user, during a procedure. In some examples, telescopic support assembly 2601 may be positioned around a shaft of endoscope 105 prior to inserting the shaft into a patient.

FIG. 26 illustrates an alternative embodiment of control system 2600. Control system 2600 may include any of the features of control system 2400 described herein, and may not include a proximal portion of base frame 1940. By removing a proximal portion of base frame 1940, control system 2600 may reduce the pitch point potential of shaft 1995 during operation of control system 2600. For example, to reduce a possible pinch point between first frame 1901 and base frame 1940, the proximal and distal section of base frame 1940 are made larger than the height of the first frame 1901 in a radial direction from the central axis 1999.

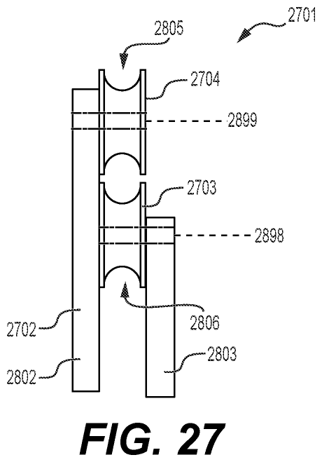

Also included in FIG. 26, an actuator assembly 2701 may be included in control system 2600. Actuator assembly 2701 may include an actuator frame 2702, a first circular member 2703, and a second circular member 2704. Referring to FIG. 27, each of circular members 2703, 2704 may be cylindrical and may include a central lumen extending longitudinally through a central longitudinal axis 2898, 2899, and each central lumen may receive a portion of actuator frame 2702. Each circular member 2703, 2704 may include a curved, radially-outward facing, relative to its longitudinal axis 2898, 2899, respectively, surface 2806, 2805. Each radially-outward facing surface 2806, 2805 may be configured to receive and substantially align with a radially-outward facing surface, relative to central longitudinal axis 1999, of shaft 1995. Actuator frame 2702 may include a first portion 2802 coupled to each of circular members 2703, 2704, and a second portion 2803 coupled to only circular member 2703 (shown in FIG. 27). In some examples, first circular member 2703 may be moveable relative to second circular member 2704. For example, first circular member 2703 may be biased towards a position in which shaft 1995 is abutting first circular member 2703 and second circular member 2704 when shaft 1995 is positioned between first circular member 2703 and second circular member 2704. First circular member 2703 (or, in other examples, second circular member 2704) may be moveable away from second circular member 2704 (or first circular member 2703, respectively) to release shaft 1995 from actuator assembly 2701. Each of circular members 2703, 2704 may be pulleys. One or more motors may be incorporated into actuator assembly 2701, and the one or more motors may drive at first circular member 2703, second circular member 2704, or both first circular member 2703 and second circular member 2704. In some examples, actuator assembly 2701 may be electronically connected to, via one or more wires or wirelessly, a control unit 199 and/or one or more handheld controllers configured to operate the one or more motors.

In some examples, each of first circular member 2703 and second circular member 2704 may be actuated simultaneously and may rotate about their respective longitudinal axis 2898, 2899 at the same rate to move shaft 1995 in a proximal or distal direction. In other examples, only one of first circular member 2703 or second circular member 2704 may be actuated and driven by a motor, and the other of first circular member 2703 and second circular member 2704 may be freely rotatable about its respective longitudinal axis 2898, 2899. In some examples, one or more of curved radially-outer surfaces 2805, 2806 may be coated in rubber or other material to increase friction between curved radially-outer surfaces 2805, 2806 and shaft 1995. By providing actuator assembly 2701, control system 2600, or any other control system discussed herein, may be positioned close the patient during a procedure, which may be more convenient to the user and may facilitate operation of control system 2600. During operation, a user may first insert shaft 1995 into actuator assembly 2701 by positioning shaft 1995 between first circular member 2703 and second circular member 2704, and the user may then proceed with an operation including any of the steps described herein in relation to control systems 100, 2900, 1900, 2600. The user may actuate actuator assembly 2701, via a control unit, remote control, or other device, to move shaft 1995 in a proximal or distal direction (into and out of a patient during a procedure).

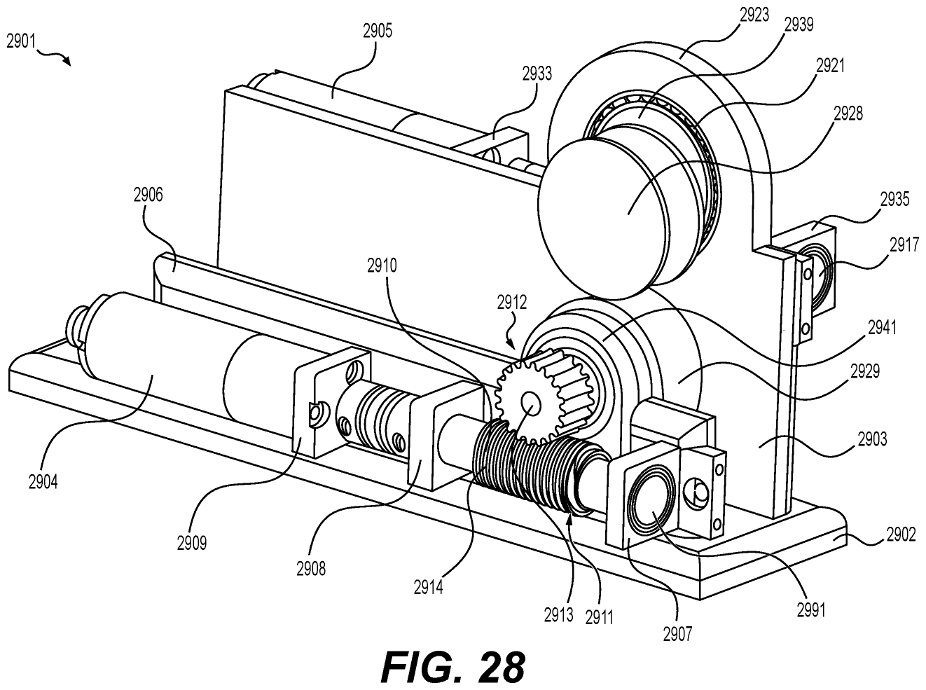

FIGS. 28-34 illustrate another embodiment of an actuator assembly 2901. Actuator assembly 2901 may have any of the features described hereinabove with regard to actuator assembly 2701, and may be used in the same manner as actuator assembly 2701 with any of the devices described herein, such as control systems 100, 2900, 1900, 2600, along with any other suitable medical devices. For example, a user may actuate actuator assembly 2901, via a control unit, remote control, or other device, to move shaft 1995 in a proximal or distal direction (into and out of a patient during a procedure), as will be described further hereinbelow.

FIG. 28 illustrates a perspective view of actuator assembly 2901 including a first circular member 2928 and a second circular member 2929. Each of first circular member 2928 and second circular member 2929 may be moveably coupled to a first frame portion 2903, and first frame portion 2903 may be substantially planar. First frame portion 2903 may be fixedly coupled to a base 2902, and first circular member 2928 and second circular member 2929 may be coupled to the same side of first frame portion 2903. A first motor 2905 may be operably coupled to first circular member 2928, and a second motor 2904 may be operably coupled to second circular member 2929. First motor 2905 may be coupled to an opposite side of first frame portion 2903 as first circular member 2928 and second circular member 2929. Second motor 2904 may be coupled to a second frame portion 2906, and second frame portion 2906 may be substantially planar and coupled to base 2904. Second motor 2904 is on the same side of first frame 2903 as first circular member 2928, and on the opposite side of first frame 2903 from first motor 2905.

Second frame portion 2906 may include a series of protrusions 2907-2909, and each protrusion 2907-2909 may include a lumen configured to receive a second drive shaft 2991. Second motor 2904 may drive (e.g., rotate) second drive shaft 2991. A second worm gear 2914 may be coupled to second drive shaft 2991, and second worm gear 2914 may be rotated by second motor 2904 and may drive (e.g. rotate) second gear 2910. Second gear 2910 may include a series of teeth 2912, and teeth 2912 may engage grooves 2911 of second worm gear 2914. A third drive shaft 2913 may be coupled to second gear 2910 and may also be coupled to second circular member 2929. When second gear 2910 is rotated by second worm gear 2914, second circular member 2929 may be rotated via third drive shaft 2913.

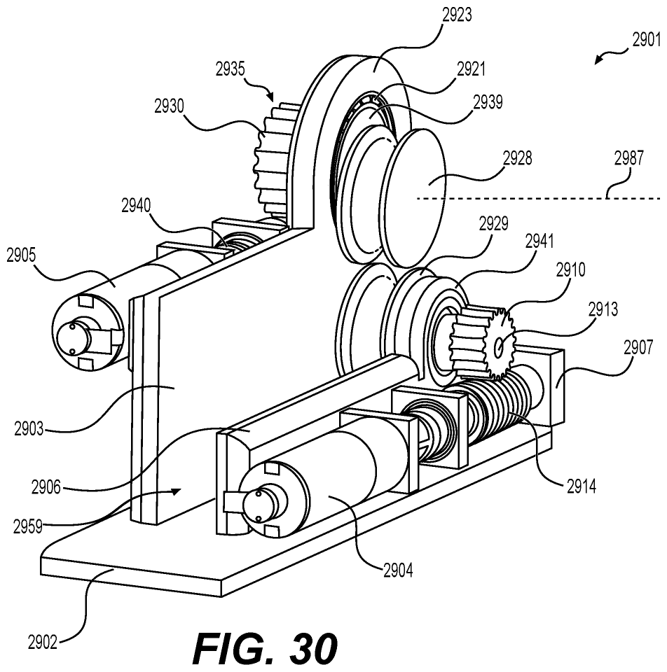

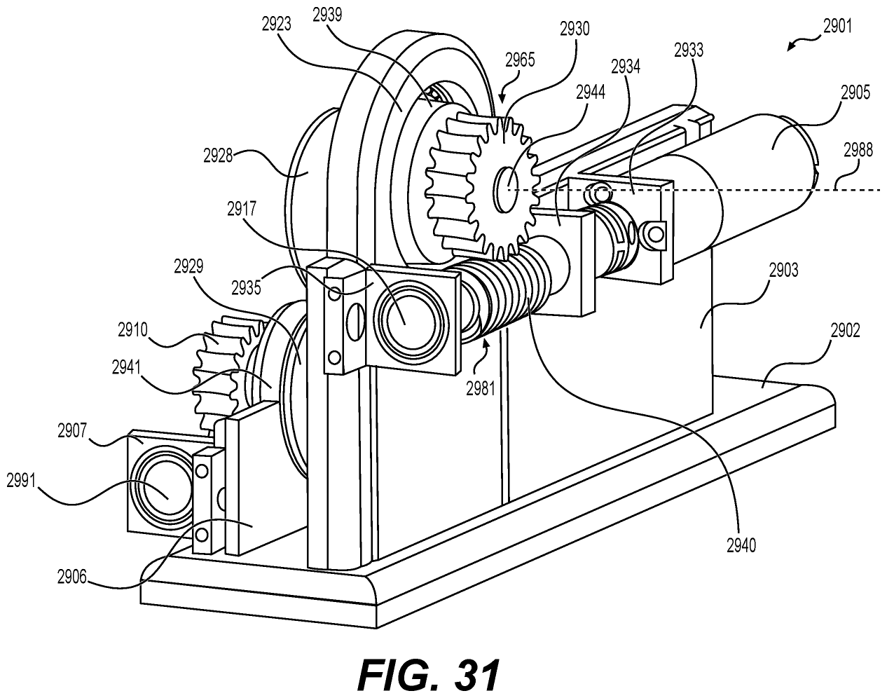

First circular member 2928 may be rotatably coupled to a circular channel 2921 in first frame portion 2903. Referring to FIGS. 30 and 31, circular channel 2921 may include a series of gears configured to engage a third circular member 2939. Third circular member 2939 may be adjacent to first circular member 2928 and third circular member 2939 may be positioned within a recess of first frame 2903, at a protruding portion 2923 of first frame 2903. First gear 2930 may be fixedly coupled to third circular member 2939 via a fourth drive shaft 2944, and fourth drive shaft 2944 may rotate about central longitudinal axis 2988. First circular member 2928 may be moveable within circular channel 2921 such that a user may adjust the distance between first circular member 2928 and second circular member 2929. As shown in FIG. 30, a central rotation axis 2987 of first circular member 2928 may be offset from central longitudinal axis 2988 of fourth drive shaft 2944.

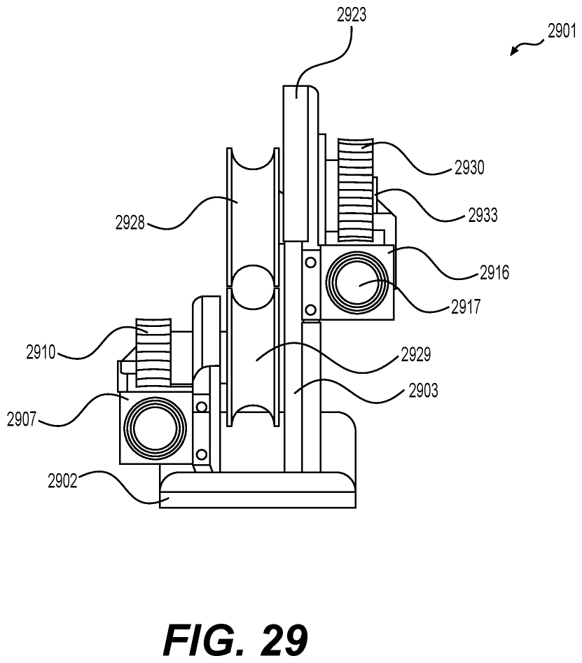

FIG. 29 illustrates a front view of actuator assembly 2901, with first circular member 2928 adjusted such that first circular member 2928 abuts second circular member 2929. As shown in FIG. 29, first circular member 2928 and second circular member 2929 are longitudinally aligned and configured to receive a shaft of a medical device. FIG. 30 illustrates a perspective, rear view of actuator assembly 2901, and shows gap 2959 between first frame portion 2903 and second frame portion 2906. Gap 2959 may be configured to receive a shaft of a medical device, and may facilitate guiding the shaft towards first circular member 2928 and second circular member 2029. Gap 2939 may help to reduce tangling in a shaft of a medical device during operation of actuator assembly 2901, for example, by helping to limit the space through with the shaft may move.

As shown in FIG. 31, first frame portion 2903 may include a series of protrusions 2933-2935, and each protrusion 2933-2935 may include a lumen configured to receive a first drive shaft 2917. First motor 2905 may drive (e.g., rotate) first drive shaft 2917. A first worm gear 2940 may be coupled to first drive shaft 2917, and first worm gear 2940 may be rotated by first motor 2905 and may drive (e.g. rotate) first gear 2930. First gear 2930 may include a series of teeth 2965, and teeth 2965 may engage grooves 2981 of first worm gear 2940. Fourth drive shaft 2944 may be coupled to first gear 2930 and may also be coupled to third circular member 2939. When first gear 2930 is rotated by first worm gear 2940, third circular member 2939 may be rotated via fourth drive shaft 2944.

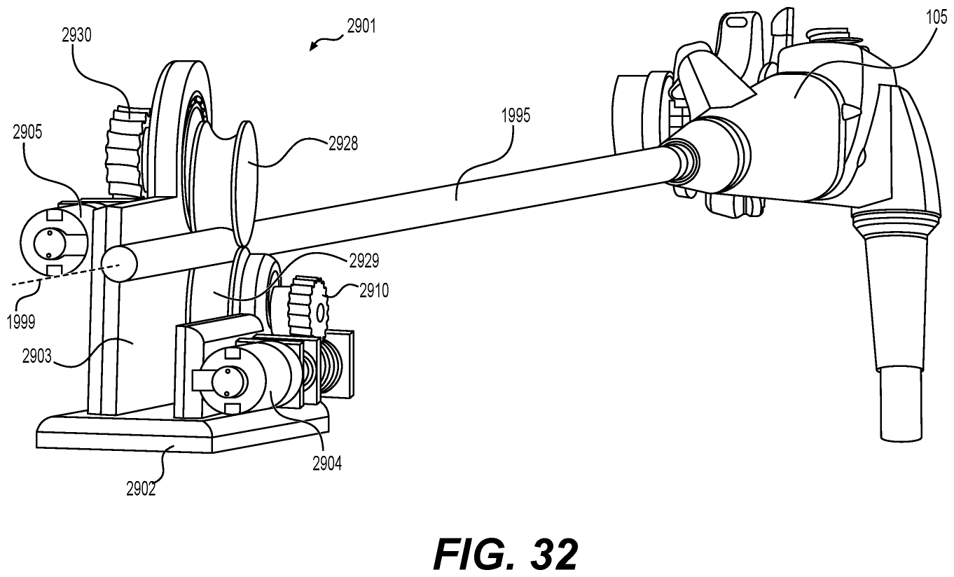

FIG. 32 illustrates a perspective view of actuator assembly 2901 coupled to shaft 1995 of endoscope 105. Each of first circular member 2928 and second circular member 2929 may be actuated simultaneously and may rotate at the same rate to move shaft 1995 in a proximal or distal direction. In other examples, only one of first circular member 2928 or second circular member 2929 may be actuated and driven by one of motors 2904, 2905, and the other of first circular member 2928 and second circular member 2929 may be freely rotatable. By providing actuator assembly 2901, control system 2600, or any other control system discussed herein, may be positioned close the patient during a procedure, which may be more convenient to the user and may facilitate operation of control system 2600. During operation, a user may first insert shaft 1995 into actuator assembly 2901 by positioning shaft 1995 between first circular member 2928 and second circular member 2928. The user may then move first circular member 2928 to a position abutting shaft 1995, and the user may then proceed with an operation including any of the steps described herein in relation to control systems 100, 2900, 1900, 2600. The user may actuate actuator assembly 2901, via a control unit, remote control, or other device, to move shaft 1995 in a proximal or distal direction (into and out of a patient during a procedure).

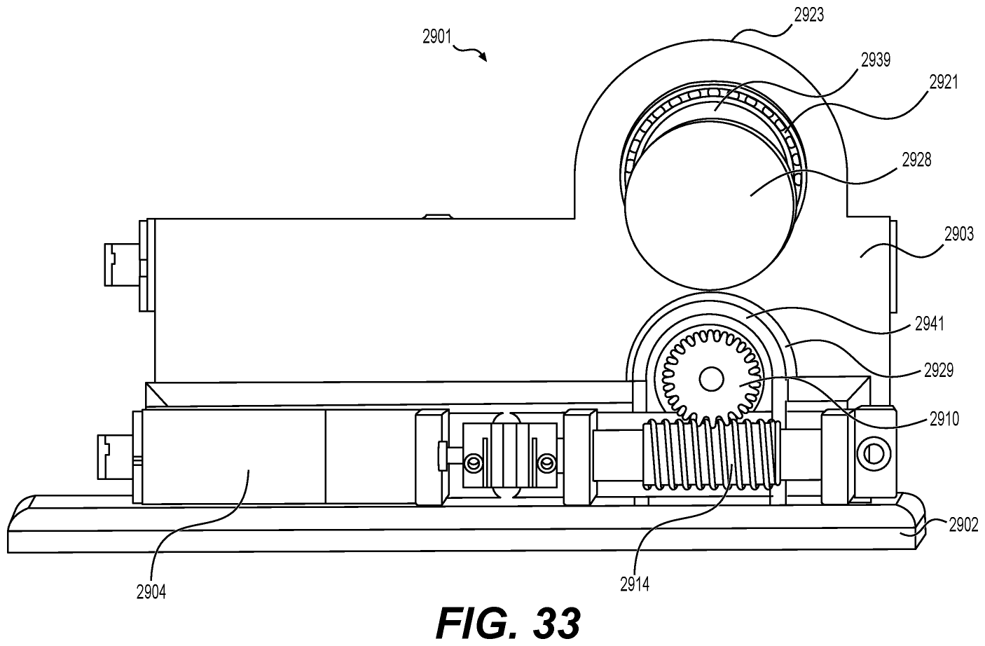

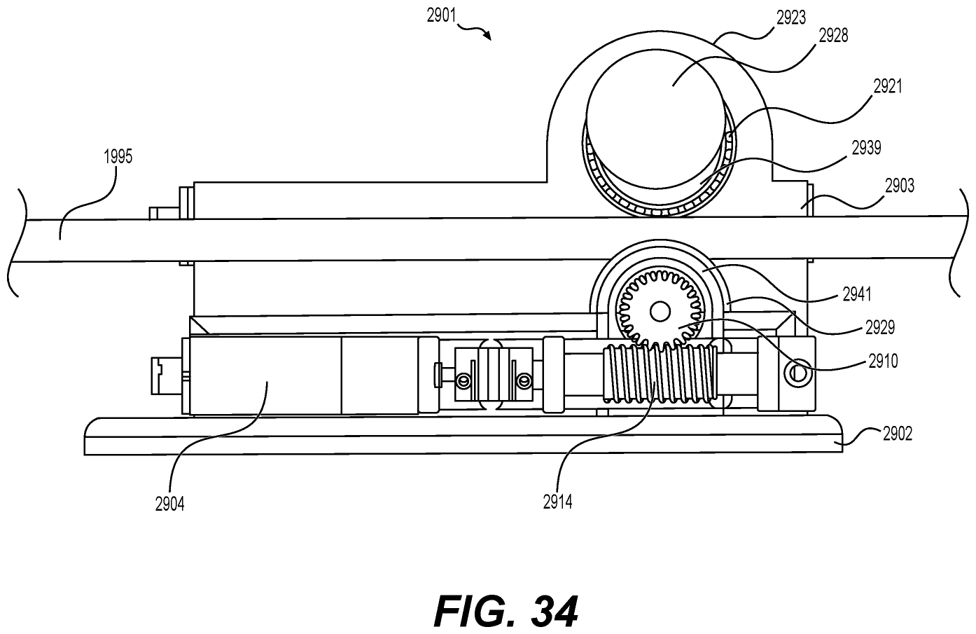

FIGS. 33 and 34 illustrate actuator assembly 2901 with first circular member 2928 in two different positions. In FIG. 33, a portion of first circular member 2928 abuts a portion of second circular member 2929. In FIG. 34, first circular member 2928 has been moved along channel 2921 to adjust the position of first circular member 2928 relative to second circular member 2929, and first circular member 2928 is spaced from second circular member 2929 to allow shaft 1995 to be positioned between first circular member 2928 and second circular member 2929. By allowing first circular member 2928 to be moveable relative to second circular member 2929, and then lockable in position during operation of actuator assembly 2901, actuator assembly 2901 may accommodate various sizes and/or shapes of shafts of medical devices.

While the disclosed methods, devices, and systems are described with exemplary reference to control unit 199 and control system 100, 2900, it should be appreciated that the disclosed embodiments may be applicable to any environment, such as a desktop or laptop computer, etc. Also, the disclosed embodiments may be applicable to any type of Internet protocol.

Embodiments of this disclosure seek to improve the operation of medical devices, such as endoscopes, and facilitate the control of movements of medical devices, such as an endoscope, during a medical procedure. As non-limiting exemplary benefits, aspects of this disclosure may reduce procedure time, improve maneuverability of medical devices, decrease procedural complications, improve patient outcomes, among other aspects.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, steps may be added or deleted to methods described within the scope of the present invention.

The above-disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

We claim:

1. A motorized control system for a medical device, comprising:
a control assembly, comprising:
a first body including a cradle assembly, wherein the cradle assembly is configured to removably couple to the medical device, the first body including:
a gear assembly,
a first motor configured to drive a first gear of the gear assembly, and
a second motor configured to drive a second gear of the gear assembly;
wherein the gear assembly is configured to receive a plurality of knobs of the medical device;
wherein the first motor is configured to drive the first gear to rotate a first knob of the plurality of knobs; and
wherein the second motor is configured to drive the second gear to rotate a second knob of the plurality of knobs.

2. The motorized control system of claim 1, further comprising a rotary drive coupled to a proximal end of the control assembly, wherein the rotary drive is configured to rotate the control assembly and the medical device about a longitudinal axis of the medical device.

3. The motorized control system of claim 2, further comprising:
a base assembly coupled to the first body; and
a rail assembly coupled to the base assembly, wherein the rail assembly includes at least one motor and is configured to move the control assembly in proximal and distal directions.

4. The motorized control system of claim 3, wherein the control assembly further comprises:
an actuator assembly comprising:
an elevator actuator configured to align with an elevator lever of the medical device;
an elevator motor; and
a third gear coupled to the elevator motor;
wherein the actuator assembly is configured to move an elevator lever of the medical device.

5. The motorized control system of claim 1, wherein the cradle assembly further comprises:
a first worm gear coupled to the first motor and engaged with the first gear; and
a second worm gear coupled to the second motor and engaged with the second gear.

6. The motorized control system of claim 5, wherein:
the first motor, the first worm gear, and the first gear are longitudinally aligned;
the second motor, the second worm gear, and the second gear are longitudinally aligned; and
the first gear is adjacent to the second gear.

7. The motorized control system of claim 1, wherein the first gear includes a series of recesses configured to align with prongs of the first knob; and wherein the second gear includes a series of recesses configured to align with prongs of the second knob.

8. The motorized control system of claim 1, wherein the control assembly is controlled by a control unit including an electronic display.

9. The motorized control system of claim 1, wherein the first body includes a first frame, a base frame, and a mounting plate.

10. The motorized control system of claim 9, wherein the mounting plate extends longitudinally in a proximal-distal direction, and wherein the first motor and the second motor are coupled to the mounting plate.

11. The motorized control system of claim 1, further comprising a telescopic support assembly.

12. The motorized control system of claim 9, wherein the first frame includes a first distal lumen and the base frame includes a second distal lumen, and wherein each of the first distal lumen and the second distal lumen are configured to receive a portion of the medical device.

13. The motorized control system of claim 1, further comprising a remote control configured to communicate with the control assembly to operate the first motor and the second motor.

14. The motorized control system of claim 1, wherein the medical device is an endoscope.

15. The motorized control system of claim 1, further comprising an actuator assembly including a first circular member and a second circular member, wherein the actuator assembly is configured to receive a shaft of the medical device.

16. A motorized control system for a medical device, comprising:
a control assembly, comprising:
a first body including a cradle assembly, wherein the cradle assembly is configured to removably couple to the medical device, the first body comprising:
a gear assembly, and
a first motor configured to drive a first gear of the gear assembly; and
a rotary drive coupled to a proximal end of the control assembly, wherein the rotary drive is configured to rotate the control assembly and the medical device about a longitudinal axis of the medical device;
wherein the gear assembly is configured to receive a first knob of the medical device; and
wherein the first motor is configured to drive the first gear to rotate the first knob.

17. The motorized control system of claim 16, wherein the control assembly further comprises:
an elevator actuator assembly comprising:
an elevator actuator configured to align with an elevator lever of the medical device, wherein the elevator actuator includes at least one biasing member;
an elevator motor; and
a third gear coupled to the elevator motor and engaged with the elevator motor;
wherein the elevator actuator assembly is configured to move an elevator lever of the medical device.

18. The motorized control system of claim 16, wherein the first gear includes a series of recesses configured to align with prongs of the first knob.

19. A motorized control system for a medical device, comprising:
a control assembly comprising:
a first body configured to removably couple to the medical device, the first body including:
a gear assembly,
a first frame rotatably coupled to a base frame; and
a first motor configured to drive a first gear of the gear assembly; and
an actuator assembly configured to move an elevator lever of the medical device, comprising:
an elevator actuator configured to align with an elevator lever of the medical device;
an elevator motor; and
a third gear coupled to the elevator motor,
wherein the gear assembly is coupled to the first frame and is configured to receive a first knob of the medical device; and
wherein the first motor is configured to drive the first gear to rotate the first knob.

20. The motorized control system of claim 19, wherein the first frame includes a first lumen and the base frame includes a second lumen longitudinally aligned with the first lumen; and wherein each of the first lumen and the second lumen is configured to receive a portion of the medical device.
